SEQUENCE LISTING (2 sequences)

SEQ ID NO: 1 - protein, 343 aa, Artificial Sequence
  Lamprey Parapinopsin Protein sequence (BAD13381.1)
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCGRNPWAEPSSESATAASTSATSVTLASAPGQVSPS

SEQ ID NO: 2 - DNA, 1032 bp, Artificial Sequence
  Lamprey Parapinopsin Nucleotide sequence (AB116380.1)
atggagaacttgacctcgctcgacctcctgcccaacggcgaggtcccgttgatgccccgctacggcttcaccatcctcgccgtgatcatggccgtgttcaccatcgcctcgctcgtgctcaacagcaccgtcgtcatcgtcaccctgcgccaccgccagctacgccacccgctcaacttctcgctcgtcaacctcgccgtggcggacctgggcgtcacggtgttcggcgccagcctcgtcgtggagaccaacgccgtcgggtacttcaacctcggccgcgtcggctgcgtcatcgaagggttcgccgtcgctttcttcggcatcgccgctctgtgcacgatcgccgtgatcgccgtcgatcgcttcgtggtggtgtgcaagccgctgggcacgctgatgttcacgcggcgccacgcgctgctgggcatcgcctgggcctggctctggtcgttcgtgtggaacacgccgccgctcttcggctggggcagctacgagctggagggcgtgcggacgtcgtgcgcgcccgactggtacagccgcgaccccgccaacgtgtcgtacattacgagctacttcgccttctgcttcgccatccccttcctcgtcatcgtggtggcgtacggccgcctcatgtggaccctccaccaggtggccaagctggggatgggcgagagcggcagcaccgccaaggcggaggcgcaggtgtcgcgcatggtggtggtcatggtggtggccttcctcgtctgctggctgccctacgcgctcttcgccatgatcgtggtgaccaagcccgacgtgtacatcgacccggtcatcgccacactgcccatgtacctgaccaagacgagcacggtctacaaccccatcatctacatcttcatgaaccgccagttccgggactgcgccgtgcccttcctgctctgcggacgcaacccctgggcagagccttcctcggagtcggcgacggcggcctccacgtcggccaccagcgtgaccctggcgtccgcgccggggcaggtctccccgagctag

(19) United States
(12) Patent Application Publication
(10) Pub. No.: US 2022/0031864 A1
(43) Pub. Date: Feb. 3, 2022

(54) QUANTITATIVE REGULATION OF A G PROTEIN SIGNALLING PATHWAY

(71) Applicant: THE UNIVERSITY OF MANCHESTER, Manchester Greater Manchester (GB)

(72) Inventors: Robert Lucas, Manchester Greater Manchester (GB); Jessica Rodgers, Manchester Greater Manchester (GB)

(73) Assignee: THE UNIVERSITY OF MANCHESTER, Manchester Greater Manchester (GB)

(21) Appl. No.: 17/277,672

(22) PCT Filed: Sep. 24, 2019
(86) PCT No.: PCT/GB2019/052685

§ 371 Date: Mar. 18, 2021

(30) Foreign Application Priority Data
Sep. 24, 2018 (GB) 1815513.5
Apr. 2, 2019 (GB) 1904631.7

(51) Int. Cl. A61K 48/00; C07K 14/46; A61P 27/02; G01N 33/50

(52) U.S. Cl. CPC A61K 48/00; C07K 14/461; A61K 38/00; G01N 33/5005; A61P 27/02

(57) ABSTRACT

The present invention relates to methods of regulating G protein activity, and related methods for the treatment of therapeutic conditions, for example retinal degeneration, by transforming a cell with a bistable opsin to activate a G protein, and provide a phototransduction response.

The present invention relates to methods of regulating G protein activity, and related methods for the treatment of therapeutic conditions, for example retinal degeneration. The present invention also relates to vectors, compositions and kits, in particular for use in a method of the invention.

### BACKGROUND

The retina of the vertebrate eye serves the same function as a film in a camera, that is receiving a visual image created by light passing through the lens and cornea of the eye. The image received is translated into chemical and electrical signals which are transmitted to the brain via the optic nerve.

The retina is a complex structure, comprising ten distinct layers of different cell types. Of these layers, it is the photoreceptor layer which is responsible for translating the incoming light into a chemical and/or electrical signal which can be read by the brain and interpreted into an image. The photoreceptor layer comprises photosensitive cells of two types, known as rod cells and cone cells. These cell types are both responsible for reacting to incoming light and producing an electrical signal, but differ in in their positioning within the retina and the type of light which they react to. Specifically, rod cells function mostly in dim light and are found predominantly in the peripheral retina. Cone cells are more reactive to bright light (i.e. daytime vision), are responsible for colour vision and are found at highest density in the central retina. The retina also contains a third, less numerous, type of photoreceptor cell—photosensitive ganglion cells—which are responsible for measuring background light, and may contribute to aspects of image processing.

The rod and cone cells of the photoreceptor layer of the retina are able to react to light and convert it to an electrical signal due to the presence of photosensitive pigments (referred to as photopigments) therein, which undergo a chemical change when the cell is exposed to light. These photopigments are G-protein-coupled receptors. The photopigments comprise a protein moiety which is coupled to a chromophoric cofactor known as retinal. Exposure to light causes an isomerisation of the retinal cofactor from a cis-retinal to a trans-retinal, which in turn causes a conformational change in the opsin protein, known as photobleaching. This is the first step in a signaling cascade which results in a signal being transmitted along the optic nerve. In order to retain photosensitivity, opsins thus need a continuous supply of cis retinal. Neither rod nor cone photoreceptor cells are able to produce cis-retinal themselves. The major source of cis-retinal in the retina is the RPE (retinal pigment epithelium) which takes all trans-retinal from bleached opsin and produces cis-retinal. In the intact retina, rod and cone cells abut the RPE allowing them access to this regenerated chromophore.

In humans, several closely related photopigments exist, known as the opsin family. In humans, these comprise 3 cone opsins which are sensitive to different wavelengths (the origin of colour vision), rhodopsin found in rod cells, and melanopsin found in ganglion cell photoreceptors. The cone opsins include LWS opsin for yellowish-green, MWS opsin for green, and SWS opsin for bluish-violet. These opsins show high sequence identity.

Conditions such as retinal dystrophies cause blindness due to destruction of the photoreceptors in the outer retina (i.e. the rods and cones). These conditions may be a result of direct damage to the photoreceptors, or photoreceptors being indirectly destroyed as a result of pathology in the retinal pigment epithelium and/or choroid. Severe visual impairment is common in advanced stages of the degeneration. These conditions are currently incurable. Retinal dystrophies can be divided into rod-cone dystrophies (also called retinitis pigmentosa), cone-rod dystrophies and macular dystrophies. In rod-cone dystrophies the rod photoreceptors degenerate resulting in a loss of peripheral vision and night vision, and frequently this is followed by cone destruction leading to a loss of central and colour vision. Conversely in the cone-rod dystrophies there is initially a loss of cone photoreceptors leading to a loss of detailed and colour vision and this is then followed by rod degeneration resulting in a loss of peripheral vision and night blindness. Both forms can result in blindness with extensive or complete loss of visual field. Another type of retinal dystrophy called macular dystrophy results in a loss of central vision, but peripheral vision is preserved.

However, despite the loss of outer retinal photoreceptors, inner retinal neurons, including bipolar cells and retinal ganglion cells, can survive and retain their ability to send visual information to the brain. These neurons therefore provide a promising niche for emerging optogenetic therapies that aim to convert them into directly visual photoreceptors and recreate the photosensitivity that has been lost with the degeneration. Several therapeutic strategies have shown promising results in attempts to replace or revive these inner retinal neurons and restore vision. Transplantation of photoreceptor cells, or their progenitor lines, is a major approach under pre-clinical study and has been shown to restore vision to blind mice at late stage of degeneration after complete loss of photoreceptors. Various attempts have been made to revive inner retinal neurons, including transforming such cells with heterologous rhodopsin to convert the cells to photoreceptor cells (WO2015/128624).

Animal opsins (light-sensitive G protein coupled receptors) can be divided into monostable and bistable opsins. Monostable visual pigments or bleaching pigments, such as rod opsin and cone opsins, bind an 11-cis retinal chromophore in their dark-adapted or inactive state. Absorption of a photon leads to photoisomerisation of the chromophore to all-trans retinal, and conformational shift in the opsin protein to active or G protein signalling state.

The opsin is then deactivated by a 2 step process (phosphorylation by a g protein kinase and binding of arrestin), which terminates G protein signalling. The all-trans chromophore is released and recycled by external mechanisms in the retinal pigment epithelium (RPE). The requirement for external deactivation mechanisms creates a relaxation period between light responses for each monostable opsin protein, and necessitates a constant supply of cis-retinal chromophore.

Bistable pigments, such as drosophila rhodopsin or lamprey parapinopsin, can bind either cis or trans-retinal chromophore. Transitions in both directions between inactive (dark-adapted) and active (G protein signalling) states are driven by light absorption. These two states can exist in equilibrium under steady illumination. Furthermore, the chromophore remains bound to the opsin protein throughout and is converted between trans and cis retinal without external recycling mechanisms.

Lamprey Parapinopsin (LPPN) is a vertebrate bistable opsin first identified by Akihisa Terakita and Mitsumasa Koyanagi at Osaka City University, Japan. Koyanagi et al ((2004) Proc. Natl. Acad. Sci. U.S.A. 101, 6687-6691. doi: 10.1073/pnas.0400819101) demonstrate that lamprey parapinopsin is a bistable opsin, with peak spectral sensitivity of 370 nm in dark adapted (inactive) state and 515 nm in G protein signalling (active) state. They also show LPPN binds both trans and cis retinal. Kawano-Yamashita et al ((2015) PLoS One. 10(10):e0141280. doi: 10.1371/journal.pone.0141280) demonstrate that the purified LPPN protein can activate transducin in response to UV light. Using Glosensor (a bioluminescent reporter of cAMP), they also show that LPPN expressed in Hek293 cells can mediate Gi signalling, inducing cAMP decreases in response to short wavelength (at blue/UV boundary) and cAMP increases in response to green light.

The present invention aims to improve the treatment of retinal degeneration, and further provide a therapy for other G protein related conditions.

### BRIEF SUMMARY OF THE DISCLOSURE

In a first aspect, there is provided a method of regulating Go protein activation in a cell, wherein the method comprises i) transforming the cell with a vector comprising a nucleic acid sequence encoding a bistable opsin, wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths of light being different; ii) expressing the nucleic acid sequence encoding the bistable opsin in the transformed cell; iii) illuminating the transformed cell expressing the bistable opsin with light comprising the first and/or second wavelength, wherein the ratio of the two wavelengths of light regulates a Go protein activation.

Therefore, the transition from the inactive state to active state is sensitive to a first wavelength of light and the transition from active to inactive state is sensitive to a second wavelength of light.

The bistable opsin in the inactive state absorbs light of a first wavelength more efficiently than the bistable opsin in the active state absorbs the light of the first wavelength. Therefore, absorption of light of the first wavelength preferentially drives transition from the inactive state to the active state. The bistable opsin in the active state absorbs light of a second wavelength more efficiently than the bistable opsin in the inactive state. Therefore, absorption of light of the second wavelength preferentially drives transition from the active state to the inactive state. The efficiency of absorption of light of a first or second wavelength may be referred to as the spectral sensitivity of the bistable opsin in either state. Therefore, it may be said that the inactive state of the bistable opsin is spectrally sensitive to a light of the first wavelength; and the active state of a bistable opsin may be spectrally sensitive to light of a second wavelength.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a second aspect, there is provided a method of quantitative regulation of G protein activation in a cell, wherein the method comprises i) transforming the cell with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light comprising the first and/or second wavelength, wherein the ratio of the two wavelengths of light regulates the amplitude of a G protein signalling cascade.

In the second aspect, the size of the G protein activation response is regulated by the ratio of (light of a first wavelength): (light of a second wavelength) used to illuminate the cell.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a third aspect, there is provided a method of regulating G protein activation in a cell, wherein the method comprises i) transforming the cell with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength wherein the ratio of the two wavelengths of light regulates a G protein signalling cascade; and wherein the time for G protein response to illumination is between 0.05 and 15 seconds.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a fourth aspect, there is provided a method for regulation of the quantity of bistable opsin in an inactive and active state in a cell, wherein transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; wherein the method comprises illuminating the cell expressing the bistable opsin with light of a first wavelength and/or light of a second wavelength, and wherein the ratio of the two wavelengths of light regulates the amount of bistable opsin in the active state.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a fifth aspect, there is provided a method of restoring vision in a subject by restoring a visual signal transduction response in a retinal cell in a subject, the method comprising i) transforming a retinal cell in the subject with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a sixth aspect, there is provided a method of treatment of retinal degeneration by restoring a visual signal transduction response in a retinal cell in a subject, the method comprising i) transforming a retinal cell in the subject with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In a seventh aspect there is provided a nucleic acid vector comprising a nucleic acid encoding i) a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; and ii) a promoter which is specific for expression in an inner retinal cell.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

In an eighth aspect of the present invention, there is provided a kit comprising i) a nucleic acid vector comprising a nucleic acid encoding a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different, and ii) a light source which emits light in the first wavelength and/or light in the second wavelength.

In an embodiment, the bistable opsin is a parapinopsin. In an embodiment, it is lamprey parapinopsin. In an embodiment, the light of the first wavelength has a wavelength of 380 to 450 nm. In an embodiment, the light of the second wavelength has a wavelength of 450 nm to 650 nm.

### BRIEF DESCRIPTION OF THE DRAWINGS

Embodiments of the invention are further described hereinafter with reference to the accompanying drawings, in which:

FIG. 1 is shows a schematic of the photochemistry of monostable and bistable pigments (extracted from Hisao Tsukamoto & Akihisa Terakita; Photochem. Photobiol. Sci., 2010, 9, 1435-1443);

FIG. 2 shows photochemistry work conducted by Koyanagi et al (Proc. Natl. Acad. Sci. U.S.A. 101, 6687-6691. doi: 10.1073/pnas.0400819101) on the purified protein to demonstrate that lamprey parapinopsin is a bistable opsin, with peak spectral sensitivity of 370 nm in dark adapted (inactive) state and 515 nm in G protein signalling (active) state. These are the wavelengths of light that optimally drive the ON and OFF response, respectively. They also show LPPN binds both trans and cis retinal.

FIG. 3 shows a live cell assay conducted by Kawano-Yamashita et al (PLoS One. 10(10):e0141280. doi: 10.1371/journal.pone.0141280) to demonstrate the LPPN protein can couple to Gi. They conduct a live cell assay using cAMP reporter Glosensor to examine lamprey parapinopsin signalling via endogenous Gi in Hek293 cells (by measuring decreases in Forskolin-elevated cAMP). They show decreases in Glosensor luminescence signal in response to UV and increases in cAMP in response to green light, consistent with Lamprey PPN Gi signalling being switched on and off. Lamprey parapinopsin can also respond repeatedly without bleaching.

FIG. 4 A. Bioluminescent resonance energy transfer (BRET) reporter schematic. Dissociation of the G-protein subunits is assayed using GRK3 fragment tagged with a nanoluciferase (NLuc). This has high affinity for free Gb/y dimer tagged with a fluorescent protein (Venus), resulting in BRET. B. Representative BRET light response. HEK293 cells expressing rod opsin and the BRET reporter (top line, filled circles) or just the reporter (bottom line, unfilled triangles) given a flash of light at time indicated by arrow. Note the time-delimited increase in Venus fluorescence (normalised to light produced by Nluc as a ‘BRET ratio’) in the opsin expressing cells.

FIG. 5—shows BRET light responses in Hek293 cells expressing either lamprey parapinopsin (LPPN, filled circles) or empty vector (No opsin, unfilled circles). Vertical lines show timing of 1 s light stimuli. Data are normalised to dark-adapted baseline. No changes in BRET ratio are observed in negative control condition.

FIG. 6—shows the predicted effective photons for the active and inactive states of lamprey parapinopsin for mixed stimuli. Although all stimuli have same total effective photons (“total”), as the ratio of blue light (405 nm) increases, the stimuli should increasingly drive the ON responses (“inactive state”) over the OFF responses (“active state”).

FIG. 7—shows the BRET light ON response for HEK293 cells expressing lamprey parapinopsin. Cells are dark adapted then flashed with 8 s of light stimulus composed of mix of blue and green light. All stimuli have same total effective photons for LPPN. B) Increasing the amount of blue light (405 nm) in the stimuli results in increasing BRET signal.

FIG. 8—shows the BRET light OFF responses for cells expressing LPPN. Cells were dark adapted then flashed with 8 s blue light (405 nm) only. After 90 s, they were flashed for 8 s with stimuli composed of a mix of blue and green light. B) Increasing relative blue results in larger BRET ratio (i.e. smaller decreases from light exposed BRET signal) normalised to dark adapted baseline.

FIG. 9—shows the BRET signal recorded for dark adapted cells (ON response—solid line, filled circles) and light-exposed cells (OFF response—dashed line, unfilled circles) after light stimulus with variable level of blue and green light. Both ON and OFF responses result in same level of BRET signal with increasing blue light—suggesting amount of G protein activation can be controlled from either direction.

FIGS. 10-10(a) shows that using BRET assay for LPPN G protein activation has a half life of 1.35 s; Response deactivation are measured with BRET assay has a half life of 8.46 s. In reality, it is probably faster (comparable to activation) and the slower speed is likely due to delay associated with BRET reporter (dissociation of GRK3 from BY dimer) (10b); For comparison Rod opsin has half life of activation of ˜6 s and half life of deactivation of ˜84 s measured using the same assay (10c);

FIG. 11 is a schematic of a Lamprey Parapinopsin-mCherry viral transgene;

FIG. 12 shows immunofluorescence against mCherry for wholemount of a retina from a Grm6Cre rd1 mouse after intraocular injection with LPPN-mCherry AAV. Retina is displayed bipolar-cell side up—small bright spots represent individually transduced bipolar cells.

FIG. 13 shows examples of 2 light-responsive channels from multi-electrode array recordings of Grm6cre rd1 retinas transduced with lamprey parapinopsin virus. Top panel shows rasters with first trials at top. Bin size=200 ms. Bottom panel shows average across trials of spike firing rate for each channel. Timing of 405 nm and 525 nm light flashes at 0 s and 22 s respectively is shown by shaded vertical bars.

FIG. 14 shows example activity of 2 channels from multi-electrode array recordings of uninjected rd1 retinas. Top panel shows rasters with first trials at top. Bin size=200 ms. Bottom panel shows average across trials of spike firing rate for each channel. Timing of 405 nm and 525 nm light flashes at 0 s and 22 s respectively is shown by shaded vertical bars.

FIG. 15 shows diverse light responses in L7Cre rd1 retinas transduced with lamprey parapinopsin-mCherry AAV.

FIG. 16 shows retinal cells expressing Lamprey parapinopsin to be excited by green light and inhibited by blue or mixed light.

FIG. 17 shows light responses where blue and mixed light cause different levels of excitation or inhibition.

FIG. 18 shows that sequential exposure to different wavelengths of light shows lamprey parapinopsin can be used to precisely control retinal activity

FIG. 19 shows example ipRGC responses to sequential wavelength stimuli

FIG. 20 shows mCherry fluorescence (bright white spots) of virally transduced cells in the retinal wholemount on the multielectrode array (example mCherry expressing cells are shown with white arrows). Individual multi-electrode array electrodes are shown as black dots

FIGS. 21 to 26 show perievent histograms (average across trials) and raster plots to flashes from dark or sequential light stimuli (21 to 24 show flashes from dark, 25 and 26 show sequential light stimuli) for retinas 1 to 4.

FIG. 27 shows the nucleic acid and amino acid sequence for LPPN.

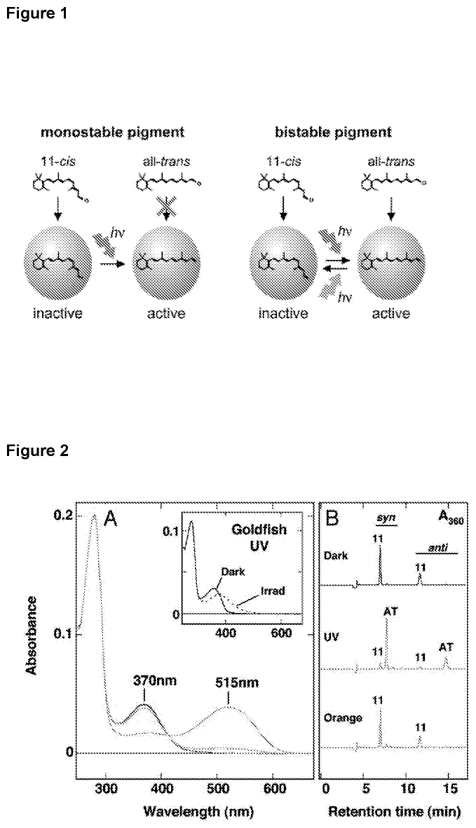

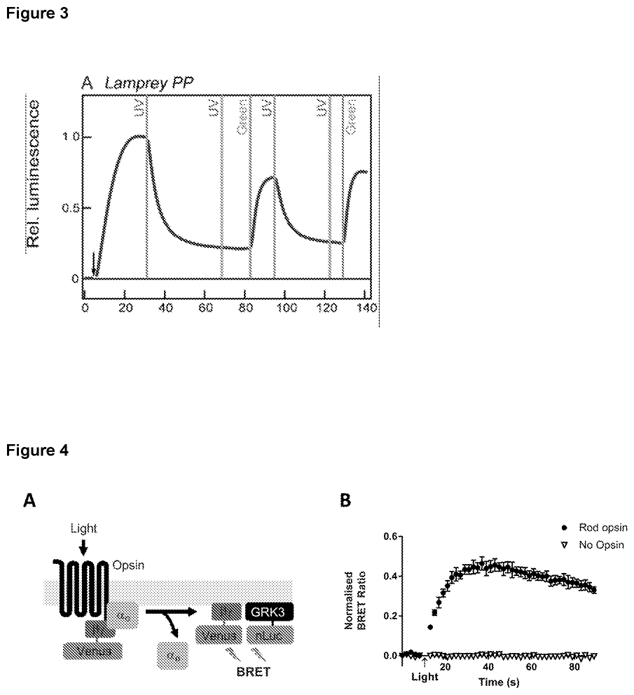

### DETAILED DESCRIPTION

The present invention relates to methods for regulating the activity of a G protein using a bistable opsin. The invention is based upon the surprising discovery that the activity of the Go protein can be regulated by providing a cell with a bistable opsin and illuminating the cell with light of a first wavelength and/or light of a second wavelength. Specifically, it has been shown by the present inventors that illuminating a cell transformed to express a bistable opsin with blue light serves to activate the Go protein. Illuminating the cell with green light serves to return the Go activity to a baseline level. In light of this discovery, the present invention provides for the first time a mechanism for control of Go activated cellular processes in a cell expressing a bistable opsin by illuminating the cell with light of a first and second wavelengths, these being different. Furthermore, the inventors have surprisingly shown that quantitative regulation of G protein activation can be achieved, by altering the ratio of light of a first wavelength (e.g. from 380-450 nm): light of a second wavelength (e.g. from 450-650 nm) which the cell is exposed to. Increasing the amount of light of the first wavelength in the light source serves to increase the amplitude of the G protein response, and conversely decreasing the amount of light of the first wavelength (and increasing the amount of light of the second wavelength) serves to decrease the amplitude of G protein activation. Even more surprisingly, the inventors have shown that the amplitude of the G protein response can be altered in near real time by altering the ratio of the first and second wavelengths of light in the light source, and that the level of G protein activation after illumination is independent of its activation state prior to illumination. Therefore, the G protein activity is directly related to the ratio of the first and second wavelengths of light in the light source, independent of its activation state prior to exposure to the light source. These findings offer for the first time a near real time and optionally quantitative, light-mediated control mechanism for G protein signalling pathways. Where desired, the present invention also enables a binary switch from a fully activated state to a baseline (non-activated state), by illuminating a cell transformed with a bistable opsin to either light of a first wavelength (e.g. from 380-450 nm) or light of a second wavelength (e.g. from 450-650 nm) for a defined period, but not to a combination of light of the first and second wavelength in the defined period. Exposure to either the first or second wavelength of light effectively switches the bistable opsin between a fully activated state and a baseline (non-activated) state. This may be referred to as on-off or binary control.

Upon illumination of a cell transformed with a bistable opsin as described herein with light of the first wavelength, the amount of bistable opsin in the active state increases. Upon steady illumination for a period of time, a point of equilibrium will be reached, with the cell comprising a higher amount of active state bistable opsin than inactive bistable opsin (which has been converted to active state). The inactive bistable opsin may eventually become depleted under steady illumination with light of the same wavelength or ratio of wavelengths.

Upon illumination of a cell transformed with a bistable opsin as described herein with light of the second wavelength, the amount of bistable opsin in the inactive state increases. Upon steady illumination for a period of time, a point of equilibrium will be reached, with the cell comprising a higher amount of inactive state bistable opsin than active bistable opsin (which has been converted to the inactive state). The active bistable opsin may eventually become depleted under steady illumination with light of the same wavelength or ratio of wavelengths.

The time taken to reach a point of equilibrium between the states may vary, and may depend upon the light intensity used. A higher light intensity may drive a faster shift toward equilibrium.

The present inventors have surprisingly found that such a bistable opsin can be used to control Go protein activation in a cell.

Thus, in a first aspect there is provided a method of regulating Go protein activation in a cell, wherein the method comprises i) transforming the cell with a vector comprising a nucleic acid sequence encoding a bistable opsin, wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light comprising the first and/or second wavelength, wherein the ratio of the two wavelengths of light regulates a Go protein activation.

In an embodiment, the first and second wavelength differ by at least 10 nm. Where the light of the first and/or second wavelength is not a single wavelength, those of the maximum output are relevant, and differ from each other by at least 10 nm.

In an embodiment, the light of a first wavelength may be from 380-450 nm for transition from the inactive state to active state. The light of the second wavelength may be from 450-650 nm for transition from an active to inactive state. Suitably, the first wavelength is about 405 nm. Suitably, the second wavelength is about 525 nm.

In an embodiment, the regulation of the Go protein comprises quantitative regulation. The light may therefore be a combination of light of a first wavelength and light of a second wavelength, at a ratio to elicit a desired Go protein signalling response.

The method may also comprise on-off or binary regulation of the Go protein, by illuminating the cell with either light of a first wavelength or light of a second wavelength for a defined period, and not illuminating the cell with a combination of light of the first and second wavelength during said defined period. Thus, the Go protein may be activated by exposing the cell to light of a first wavelength; and the Go protein activity may be terminated by exposing the cell to light of a second wavelength.

In an embodiment, the bistable opsin is a parapinopsin, for example lamprey parapinopsin.

In an embodiment, the bistable opsin is not naturally expressed in the cell to be transformed.

In an embodiment, the step of illuminating the cell comprises exposing the cell to the light for a defined time interval, after which the illumination is stopped. The method may comprise illuminating the cell with light of a first wavelength or light of a second wavelength for two or more discrete time intervals.

In an embodiment, a method of the first aspect relates to a method for regulation of a Go protein in a retinal cell, wherein the bistable opsin is lamprey parapinopsin. In a suitable embodiment, the cell is an inner retinal cell such a bipolar cell, suitably an ON bipolar cell.

The bistable opsin may be provided in a vector, suitably a viral vector, such as an AAV vector.

In an embodiment, a method of the first aspect relates to a method for regulation of a Go protein in a brain, nerve or heart cell, wherein the bistable opsin is lamprey parapinopsin. The bistable opsin may be provided in a vector, suitably a viral vector, such as an AAV vector.

The first aspect also provides for the use of a vector comprising a nucleic acid sequence encoding a bistable opsin, wherein the bistable opsin has an inactive and an active state, and transition in both directions between inactive and active states are driven by light absorption in a method of regulating Go protein activation in a cell. The method may be as defined in the first aspect.

In a second aspect, there is provided a method of quantitative regulation of G protein activation in a cell, wherein the method comprises i) transforming the cell with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light comprising the first and/or second wavelength, wherein the ratio of the two wavelengths of light regulates the amplitude of a G protein signalling cascade.

In the second aspect, the size of the G protein activation response is regulated by the ratio of light of a first wavelength: light of a second wavelength used to illuminate the cell.

In an embodiment, the bistable opsin is a parapinopsin, for example lamprey parapinopsin.

In an embodiment the G protein is a Go or Gi protein.

In an embodiment, the bistable opsin is not naturally expressed in the cell to be transformed. Suitably, the cell may be a retinal ganglion cell. Suitably, the cell may be an inner retinal cell, more suitably a bipolar cell, such as an ON or OFF bipolar cell. Alternatively, the cell may be a brain, nerve or heart cell.

In an embodiment, the first and second wavelength differ by at least 10 nm. Where the light of the first and/or second wavelength is not a single wavelength, those of the maximum output are relevant, and differ from each other by at least 10 nm.

In an embodiment, the light of a first wavelength may be from 380-450 nm for driving transition from the inactive state to active state. The light of the second wavelength may be from 450-650 nm for driving transition from an active to inactive state. Suitably, the first wavelength is about 405 nm. Suitably, the second wavelength is about 525 nm.

In an embodiment, the light is a combination of light of a first wavelength and light of a second wavelength.

In an embodiment, the light may be a combination of light of a first wavelength as described herein, and light of a second wavelength as described herein. Suitably, the light may be a combination of light of a first wavelength from 380-450 nm and light of a second wavelength from 450-650 nm. In an embodiment, the step of illuminating the cell comprises exposing the cell to the light for a defined time interval, after which the illumination is stopped. The method may comprise illuminating the cell with light of a first wavelength and/or light of a second wavelength for two or more discrete time intervals.

The method may also comprise qualitative regulation of the Go protein, by exposing the cell to either light of a first wavelength or light of a second wavelength. Thus, the Go protein may be activated by exposing the cell to light of a first wavelength; and the Go protein activity may be terminated by exposing the cell to light of a second wavelength.

In an embodiment, a method of the second aspect relates to quantitative regulation of a Go protein, wherein the bistable opsin is Lamprey parapinopsin and the light of the first wavelength is from 380-450 nm and light of a second wavelength from 450-650 nm.

In an embodiment, a method of the second aspect relates to quantitative regulation of a Go protein in a retinal cell, wherein the bistable opsin is lamprey parapinopsin. In a suitable embodiment, the cell is an inner retinal cell such as a bipolar cell, suitably an ON bipolar cell. The bistable opsin may be provided in a vector, suitably a viral vector, such as an AAV vector.

In an embodiment, a method of the second aspect relates to quantitative regulation of a Go protein in a brain, nerve or heart cell, wherein the bistable opsin is lamprey parapinopsin. The bistable opsin may be provided in a vector, suitably a viral vector, such as an AAV vector.

The second aspect also provides for the use of a vector comprising a nucleic acid sequence encoding a bistable opsin, wherein the bistable opsin has an inactive and an active state, and transition in both directions between inactive and active states are driven by light absorption in a method of regulating Go protein activation in a cell. The method may be as defined in the first aspect.

In a third aspect, there is provided a method of regulating G protein activation in a cell, wherein the method comprises i) transforming the cell with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength wherein the ratio of the two wavelengths of light regulates a Go protein signalling cascade; and wherein the time for G protein response to illumination is between 0.05 and 15 seconds.

The third aspect is based upon the observation by the present inventors that the G protein signalling response is significantly faster than observed in the prior art, and near real time activation and termination of a G protein signal can be achieved. Compared to the prior art showing a response time of minutes, the present inventors have shown that activation and termination of G protein signalling can be achieved much faster, within seconds of illumination. The reduction or elimination of the time lag improves the temporal resolution in opsin based therapies.

In an embodiment, the G protein is Gi or Go protein.

In an embodiment, the bistable opsin is a parapinopsin, for example lamprey parapinopsin.

In an embodiment, the first and second wavelength differ by at least 10 nm. Where the light of the first and/or second wavelength is not a single wavelength, those of the maximum output are relevant, and differ from each other by at least 10 nm.

In an embodiment, the light of a first wavelength may be from 380-450 nm for driving transition from the inactive state to active state. The light of the second wavelength may be from 450-650 nm for driving transition from an active to inactive state. Suitably, the first wavelength is about 405 nm. Suitably, the second wavelength is about 525 nm.

In an embodiment, the light is a combination of light of a first wavelength and light of a second wavelength.

In an embodiment, the light may be a combination of light of a first wavelength as described herein, and light of a second wavelength as described herein. Suitably, the light may be a combination of light of a first wavelength from 380-450 nm and light of a second wavelength from 450-650 nm. In an embodiment, the step of illuminating the cell comprises exposing the cell to the light for a defined time interval, after which the illumination is stopped. The method may comprise illuminating the cell with light of a first wavelength and/or light of a second wavelength for two or more discrete time intervals.

The method may also comprise on-off or binary regulation of the Go protein, by illuminating the cell with either light of a first wavelength or light of a second wavelength for a defined period, and not illuminating the cell with a combination of light of the first and second wavelength during said defined period. Thus, the Go protein may be activated by exposing the cell to light of a first wavelength; and the Go protein activity may be terminated by exposing the cell to light of a second wavelength. Thus, the Go protein may be activated by exposing the cell to light of a first wavelength; and the Go protein activity may be terminated by exposing the cell to light of a second wavelength.

In an embodiment, a method of the third aspect relates to a method for regulating G protein activation in a retinal cell, wherein the bistable opsin is lamprey parapinopsin. In a suitable embodiment, the cell is an inner retinal cell such as a bipolar cell, suitably an ON bipolar cell. The bistable opsin may be provided in a vector, suitably a viral vector, such as an AAV vector.

In an embodiment, a method of the third aspect relates to a method for regulating G protein activation in a brain, nerve or heart cell, wherein the bistable opsin is lamprey parapinopsin. The bistable opsin may be provided in a vector, suitably a viral vector, such as an AAV vector.

The third aspect also provides for the use of a vector comprising a nucleic acid sequence encoding a bistable opsin, wherein the bistable opsin has an inactive and an active state, and transition in both directions between inactive and active states are driven by light absorption in a method of regulating G protein activation in a cell, wherein the time for G protein response to illumination is between 0.05 and 15 seconds. The method may be as defined in the third aspect.

In a fourth aspect, there is provided a method for quantitative regulation of the quantity of bistable opsin in an inactive and active state in a cell, wherein transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; wherein the method comprises illuminating the cell expressing the bistable opsin with light of a first wavelength and/or light of a second wavelength, and wherein the ratio of the two wavelengths of light regulates the amount of bistable opsin in the active state. In an embodiment, the method comprises transforming the cell with a vector comprising a nucleic acid sequence encoding the bistable opsin and expressing the bistable opsin in the transformed cell.

In an embodiment, the bistable opsin is a parapinopsin, for example lamprey parapinopsin.

In an embodiment, the first and second wavelength differ by at least 10 nm. Where the light of the first and/or second wavelength is not a single wavelength, it is the wavelengths having the maximum output which are relevant, and differ from each other by at least 10 nm.

In an embodiment, the light of a first wavelength may be from 380-450 nm for driving transition from the active state to inactive state. The light of the second wavelength may be from 450-650 nm for driving transition from an inactive to active state. Suitably, the first wavelength is about 405 nm. Suitably, the second wavelength is about 525 nm.

In an embodiment, the light is a combination of light of a first wavelength and light of a second wavelength.

In an embodiment, the light may be a combination of light of a first wavelength as described herein, and light of a second wavelength as described herein. Suitably, the light may be a combination of light of a first wavelength from 380-450 nm and light of a second wavelength from 450-650 nm. In an embodiment, the step of illuminating the cell comprises exposing the cell to the light for a defined time interval, after which the illumination is stopped. The method may comprise illuminating the cell with light of a first wavelength and/or light of a second wavelength for two or more discrete time intervals.

The method may also comprise on-off or binary regulation of the Go protein, by illuminating the cell with either light of a first wavelength or light of a second wavelength for a defined period, and not illuminating the cell with a combination of light of the first and second wavelength during said defined period. Thus, the Go protein may be activated by exposing the cell to light of a first wavelength; and the Go protein activity may be terminated by exposing the cell to light of a second wavelength. Thus, the transition from inactive state to an active state may be driven by exposing the cell to light of a first wavelength; and the reverse transition may be driven by exposing the cell to light of a second wavelength.

In an embodiment, a method of the fourth aspect relates to quantitative regulation of the ON-OFF response of a bistable opsin in a retinal cell, wherein the bistable opsin is lamprey parapinopsin. In a suitable embodiment, the cell is an inner retinal cell such as an ON bipolar cell. The bistable opsin may be provided in a vector, suitably a viral vector, such as an AAV vector.

In an embodiment, a method of the fourth aspect relates to a method for quantitative regulation of the quantity of bistable opsin in an inactive and active state in a brain, nerve or heart cell, wherein the bistable opsin is lamprey parapinopsin. The bistable opsin may be provided in a vector, suitably a viral vector such as an AAV vector.

The fourth aspect also provides for the use of a vector comprising a nucleic acid sequence encoding a bistable opsin, wherein the bistable opsin has an inactive and an active state, and transition in both directions between inactive and active states are driven by light absorption in a method of regulation of the quantity of bistable opsin in an inactive and active state in a cell. The method may be as defined in the fourth aspect.

In a fifth aspect, there is provided a method of restoring vision in a subject by restoring a visual signal transduction response in a retinal cell in a subject, the method comprising i) transforming a retinal cell in the subject with a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell.

In an embodiment, the bistable opsin is a parapinopsin. In an embodiment, it is lamprey parapinopsin.

In an embodiment, the bistable opsin is a parapinopsin, for example lamprey parapinopsin.

In an embodiment the G protein is a Go or Gi protein.

In an embodiment, the first and second wavelengths differ by at least 10 nm. Where the light of the first and/or second wavelength is not a single wavelength, those of the maximum output are relevant, and differ from each other by at least 10 nm.

In an embodiment, the light of a first wavelength may be from 380-450 nm for driving transition from the inactive state to active state. The light of the second wavelength may be from 450-650 nm for driving transition from an active to inactive state. Suitably, the first wavelength is about 405 nm. Suitably, the second wavelength is about 525 nm.

In an embodiment, the light is a combination of light of a first wavelength and light of a second wavelength.

In an embodiment, the light may be a combination of light of a first wavelength as described herein, and light of a second wavelength as described herein. Suitably, the light may be a combination of light of a first wavelength from 380-450 nm and light of a second wavelength from 450-650 nm. In an embodiment, the step of illuminating the cell comprises exposing the cell to the light for a defined time interval, after which the illumination is stopped. The method may comprise illuminating the cell with light of a first wavelength and/or light of a second wavelength for two or more discrete time intervals.

The method may also comprise on-off or binary regulation of the Go protein, by illuminating the cell with either light of a first wavelength or light of a second wavelength for a defined period, and not illuminating the cell with a combination of light of the first and second wavelength during said defined period. Thus, the Go protein may be activated by exposing the cell to light of a first wavelength; and the Go protein activity may be terminated by exposing the cell to light of a second wavelength. Thus, the Go protein may be activated by exposing the cell to light of a first wavelength; and the Go protein activity may be terminated by exposing the cell to light of a second wavelength.

In an embodiment, a method of the second aspect relates to quantitative regulation of a Go protein in a retinal cell, wherein the bistable opsin is lamprey parapinopsin. In a suitable embodiment, the cell is an inner retinal cell such as a bipolar cell, suitably an ON bipolar cell. The bistable opsin may be provided in a vector, suitably a viral vector such as an AAV vector. In a suitable embodiment, the light of the first wavelength is about 405 nm. In a suitable embodiment, the light of the second wavelength is about 525 nm.

The fifth aspect also relates to a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different, for use in a method of restoring vision. The method may be as defined in the fifth aspect.

In a sixth aspect, there is provided a method of treatment of retinal degeneration by restoring a visual signal transduction response in a retinal cell in a subject, the method comprising i) transforming a retinal cell in the subject with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell.

In an embodiment, the bistable opsin is a parapinopsin, for example lamprey parapinopsin.

In an embodiment the G protein is a Go or Gi protein.

In an embodiment, the first and second wavelength differ by at least 10 nm. Where the light of the first and/or second wavelength is not a single wavelength, those of the maximum output are relevant, and differ from each other by at least 10 nm.

In an embodiment, the light of a first wavelength may be from 380-450 nm for driving transition from the inactive state to active state. The light of the second wavelength may be from 450-650 nm for driving transition from an active to inactive state. Suitably, the first wavelength is about 405 nm. Suitably, the second wavelength is about 525 nm.

In an embodiment, the light is a combination of light of a first wavelength and light of a second wavelength.

In an embodiment, the light may be a combination of light of a first wavelength as described herein, and light of a second wavelength as described herein. Suitably, the light may be a combination of light of a first wavelength from 380-450 nm and light of a second wavelength from 450-650 nm. In an embodiment, the step of illuminating the cell comprises exposing the cell to the light for a defined time interval, after which the illumination is stopped. The method may comprise illuminating the cell with light of a first wavelength and/or light of a second wavelength for two or more discrete time intervals.

The method may also comprise on-off or binary regulation of the Go protein, by illuminating the cell with either light of a first wavelength or light of a second wavelength for a defined period, and not illuminating the cell with a combination of light of the first and second wavelength during said defined period. Thus, the Go protein may be activated by exposing the cell to light of a first wavelength; and the Go protein activity may be terminated by exposing the cell to light of a second wavelength. Thus, the Go protein may be activated by exposing the cell to light of a first wavelength; and the Go protein activity may be terminated by exposing the cell to light of a second wavelength.

In an embodiment, a method of the sixth aspect relates to quantitative regulation of a Go protein in a retinal cell, wherein the bistable opsin is lamprey parapinopsin. In a suitable embodiment, the cell is an inner retinal cell such as a bipolar cell, suitably an ON bipolar cell. The bistable opsin may be provided in a vector, suitably a viral vector such as an AAV vector. In a suitable embodiment, the light of the first wavelength is about 405 nm. In a suitable embodiment, the light of the second wavelength is about 525 nm.

The sixth aspect also relates to a vector comprising a nucleic acid sequence encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different, for use in a method of treating retinal degeneration. The method may be as defined in the sixth aspect.

In a seventh aspect there is provided a nucleic acid vector comprising a nucleic acid encoding i) a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; and ii) a promoter which is specific for expression in an inner retinal cell.

In an embodiment, the bistable opsin is a parapinopsin, for example lamprey parapinopsin.

In an embodiment, the vector is a viral vector, suitably AAV.

In an embodiment, the promoter is specific for bipolar cells, suitably ON bipolar cells. In an embodiment, the promoter is grm6, or a fragment or a derivative thereof. The promoter may be a synthetic promoter derived from a naturally occurring grm6 promoter. The promoter may be a grm6 enhancer-SV40 fusion.

The invention provides a vector for use in a method of restoring vision and/or treating retinal degeneration. The invention also provides a vector as described herein for use in the treatment of a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability, Parkinsons disease, Alzheimer's disease, schizophrenia and heart disease. The invention also provides a vector as described herein for use in a method of restoring vision.

The vector may be provided as a suitable composition, for example an injectable liquid, for use in a method of restoring vision and/or treating retinal degeneration. The invention also provides a composition comprising a vector as defined herein for use in a method of treating a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability, Parkinsons disease, Alzheimer's disease, schizophrenia and heart disease. The invention also provides a composition as described herein for use in a method of restoring vision.

In an eighth aspect of the present invention, there is provided a kit comprising i) a nucleic acid vector comprising a nucleic acid encoding a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different, and ii) a light source which emits light in the first wavelength and/or light in the second wavelength.

In an embodiment, the bistable opsin is a parapinopsin, for example lamprey parapinopsin.

In an embodiment, the nucleic acid is provided in a vector, where the vector may be as defined in the seventh aspect. The vector may be a viral vector, such as an AAV vector.

The invention also provides a kit as described herein for use in a method of treating a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability, Parkinsons disease, Alzheimer's disease, schizophrenia and heart disease. The invention also provides a kit for use in a method of restoring vision.

### Definitions

G Proteins & Signalling Pathways

A signalling cascade initiated by an opsin is generally referred to as phototransduction. Thus, phototransduction may be defined as the intracellular steps linking light absorption by an opsin with a change in cell physiology. The intracellular steps may be referred to as a signalling cascade initiated by light absorption by an opsin, and resulting in a change in cell physiology. A signalling cascade may be part of a signalling pathway involving multiple cells, suitably of different types, each responsible for one or more steps of the pathway. Therefore, the opsin-mediated cell response may trigger cell responses in multiple cell types to form an opsin-mediated cellular pathway. The steps of phototransduction as defined herein may include the initial step of light absorption and/or the change in cell physiology.

In phototransduction, an opsin couples to a G protein. In a naturally occurring cascade, the opsin may couple to a G protein naturally present in the cell. In an artificial cascade, for example as described herein, an opsin heterologously expressed in a cell may couple to a G protein naturally present or native to said cell. Alternatively, a suitable G protein may be co-expressed in the cell. Suitable G proteins to which an opsin may couple in a cell are Go and/or Gi. The Go mediated signalling cascade is responsible for a key synaptic transmission event in the retina, from rod and cone photoreceptors to ON bipolar cells.

Phototransduction is an opsin initiated signalling pathway which results in a change in physiology in a cell type selected from the group consisting of a heart cell, endocrine cell, nerve cell, brain cell, or retinal cell.

Herein, a G protein includes any G protein which naturally couples to a bistable opsin as defined herein.

By G protein activation is meant initiation of a signalling cascade mediated by said G protein. For example in the case of Go, activation means initiation of the signalling cascade. By initiation of a signalling cascade may mean the first cellular event following absorption of light by an opsin and activation of the opsin. This may include for example activation or inactivation of the protein which is immediately downstream of the opsin in the cascade. G protein activation may further include one or more steps of the signalling cascade downstream of activation or inactivation of the protein which is immediately downstream of the opsin, up to and including a change in cell physiology.

Visual signal transduction as used herein refers to an opsin mediated network or pathway between synapses. A visual signal transduction pathway is a pathway associated with the function of the eye. Visual signal transduction may therefore include any opsin mediated signalling cascade which is part of a network or pathway which affects vision. A visual signal transduction response as referred to herein may be a downstream event in a signalling pathway which affects vision. Therefore, a visual signal transduction response is an opsin mediated activity of the visual pathway. The pathway driven by heterologous expression of a bistable opsin in retinal cells (suitably inner retinal cells) as described herein can activate the visual pathway to compensate for degeneration of rod or cone cells and consequent absence of the original opsins driving the pathway. A visual signal transduction response may be transmission of a signal from an inner retinal cell to a nerve cell, and/or physiological responses of the eye for example a pupillary light reflex, response to changing light conditions such as flicker, physical response of the subject to changing light conditions or scenes.

Regulation of the G protein signalling event using a bistable opsin and illumination with a first and second wavelength of light as described herein serves to regulate the signalling cascade. The invention enables the amplitude of the G protein response to be controlled, by altering the ratio of the first and second wavelength of light used to illuminate the cell. For example, where the G protein is a Go protein, controlling the ratio of light by altering the ratio of first and second wavelength of light emitted by the light source and therefore controlling the amplitude of Go protein activation enables the user to tune the phototransduction response to their individual needs.

Other signalling cascades mediated by G proteins which are naturally coupled to a bistable opsin may be targeted using the methods described herein. Loss or partial loss of the Go protein signalling cascade has been shown to be linked with conditions such as Parkinsons Disease, Alzheimer's disease, schizophrenia and heart conditions.

By amplitude or extent of G protein activation means the amount or degree of G protein response in a cell. G protein activation in a cell is not binary. By this it is meant that there is a range of the extent of activation of the G protein between the activated and inactivated states. The size of the signalling event can therefore vary, and can be quantified.

The size of the G protein response to activation can be measured using known methods available in the art, for example:

1) BRET and FRET assays (as described herein, and similar methods involving tagging and measuring interaction of different proteins. Specifically, the G-protein coupled receptor and G protein alpha unit are tagged with fluorescent and bioluminescent proteins (Gales et al Nature Methods volume 2, pages 177-184 (2005)); or measuring interaction of YFP tagged G protein gamma subunit with CFP tagged G protein subunit Van Unen et al PLOS (https://doi.org/10.1371/journal.pone.0146789);

2) GTPγS binding assay involving measuring the amount of GTPyS [35S] bound to G alpha subunit protein before and after GPCR activation (FEBS Letters, Volume 439, Issues 1-2, 13 Nov. 1998, Pages 110-114);

3) Fluorescent/Bioluminescent reporters of secondary messenger (downstream from G protein activation)—these include the Glosensor reporter from Promega (Buccioni et al Purinergic Signal. 2011 December; 7(4): 463-468) and cAMP nanolanterns (Saito et al Nat Commun. 2012 Dec. 11; 3: 1262), and can be combined with G protein chimeras to examine G protein selectivity (Ballister et al BMC Biology 201816:10); and

4) Immunoprecipitation assays based on antibody labelling (https://www.discoverx.com/products-applications/camp-assays; https://www.abcam.com/camp-assay-kit-direct-immunoassay-ab65355.html; https://www.thermofisher.com/order/catalog/product/4412183).

Regulation of the G protein signalling event using a bistable opsin and illumination with a first and second wavelength of light as described herein serves to regulate the signalling cascade. The invention enables the amplitude of the G protein response to be controlled, by altering the ratio of the first and second wavelength of light used to illuminate the cell. For example, where the G protein is a Go protein, controlling the ratio of light by altering the ratio of first and second wavelength of light emitted by the light source and therefore controlling the amplitude of Go protein activation enables the user to tune the phototransduction response to their individual needs.

Herein, a method of regulating G protein activation in a cell may comprise the step of observing, monitoring and/or measuring an output of G protein activation. Such output may be a change in physiology of the transformed cell, or may be an output resulting from a downstream signalling pathway initiated by the G protein activation in a different cell. Suitable methods for observing, monitoring and/or measuring an output of G protein activation as described herein will be known in the art. Other signalling cascades mediated by G proteins which are naturally coupled to a bistable opsin may be targeted using the methods described herein. Loss or partial loss of the Go protein signalling cascade has been shown to be linked with conditions such as Parkinsons Disease, Alzheimer's disease, schizophrenia and heart conditions.

In an embodiment, the G protein may be native to the cell to be transformed. Therefore, in such an embodiment, the bistable opsin introduced into the cell couples to a native G protein of the cell, to initiate the G protein signalling cascade upon activation by illumination as described herein. For example, lamprey parapinopsin expressed heterologously in an inner retinal cell will activate the endogenous or native Go signalling cascade to initiate the photo transduction pathway. Similarly, Gi activity results in a decrease in cAMP via inhibition of adenylyl cyclase in a variety of different cell types, which express Gi.

In an embodiment, the present invention may comprise providing to the cell a nucleic acid encoding a G protein for coupling to the heterologous bistable opsin, in order to initiate a signalling cascade. The signalling cascade may be one which is not natively active in the host cell. Where a G protein is foreign to the cell, it is suitably a G protein which natively couples with the bistable opsin introduced into the cell. For example, where a lamprey parapinopsin is introduced into a cell, a suitable G protein may be Gi or Go.

In an embodiment, the present invention may comprise providing to the cell a nucleic acid encoding a member of a G protein signalling cascade. By providing a member of a G protein signalling cascade which is not natively present in the cell, the physiology of the cell can be altered upon expression and activation of the heterologous bistable opsin. The G protein is provided for coupling to the heterologous bistable opsin, in order to initiate a signalling cascade. The member of a G protein signalling cascade may be one which is not natively active in the host cell.

Therefore, there may be provided in the present invention a vector comprising a nucleic acid sequence encoding a G protein and/or one or more members of a G protein signalling cascade. The vector may be the same or different to the vector comprising a nucleic acid sequence encoding the bistable opsin. Suitable regulatory sequences and vectors may be provided on the vector, as described herein in relation to the bistable opsin. In the present invention, a vector encoding a G protein and/or one or more members of a G protein signalling cascade is provided for use in combination with a vector comprising a nucleic acid sequence encoding a bistable opsin as described herein.

Regulation

Herein, regulation of a G protein signalling response or a bistable opsin transition between states means that light of the first and second wavelengths may be used to control the timing and extent (amplitude) of the response. In turn, regulation serves to control the temporal and quantitative response of the downstream signalling pathway.

Regulation of the response may be quantitative. By quantitative is meant that the amount, or level of the response can be controlled. It has been shown by the present inventors that the amplitude of G protein signalling response driven by a bistable opsin is directly related to the proportion of light of the first and second wavelength. Altering the ratio allows the cellular response to be altered. For example, using only light of a first wavelength or a higher proportion thereof enables a higher amplitude of G protein signalling, for example exposure to light of a first wavelength may drive up to twice or more response amplitude of that produced by a light comprising an equal mixture of first and second wavelengths.

For lamprey parapinopsin, the maximum response is an increase of from 1.6 fold from baseline, under light of the first wavelength for illumination, using a method known in the art, for example as described at page 21, and most suitably a BRET assay.

The methods of the invention enable real time and quantitative G protein regulation. Thus, illumination of a cell transformed with a bistable opsin according to a method of the invention can be used to regulate the degree of G protein activation in real time, wherein the degree of activation can be changed as the wavelength of light used for illumination is changed between a first wavelength and a second wavelength, and/or the ratio of the first and second wavelengths as defined herein used to illuminate a cell is changed.

Additionally, the response may be binary, or an ON-OFF response, when a cell transformed to express a bistable opsin as described herein is illuminated with light of a first or second wavelength for a defined period, but not a combination of light of the first and second wavelength. When a cell transformed to express a bistable opsin is illuminated, or exposed to, light of a first wavelength for a defined period the bistable opsin will be activated. Illumination with light of the second wavelength then returns the activity level of the bistable opsin to a baseline level. Thus, switching between light of the first wavelength and light of the second wavelength for illumination in a defined time period can be used to switch the bistable opsin between a fully activated state and a baseline state.

Bistable Opsin

Herein, a bistable opsin refers to an opsin which has an inactive and an active state, distinguished by an alteration in the conformational state of the protein. In a bistable opsin of the invention, the relationship between wavelength of light and likelihood of photon absorption differs between the active and inactive states. Therefore, one direction of transition between active and inactive states may preferentially be driven by light of a certain wavelength, and the other direction may be preferentially driven by light of a second, different wavelength.

The two states, inactive and active, are thermally stable states.

The active state is a G protein signalling state, and may be referred to as such. The activity of the bistable opsin upon exposure to light of a first wavelength only (and not to combination of light of a first and second wavelength) for a defined period may be referred to as maximum activity of the bistable opsin. The inactive state is the state in which the associated G protein is not activated, and is also referred to as the dark-adapted state. The activity upon exposure to only light of the second wavelength for a defined period may be the inactive state, or baseline state, where there is no (or only minimal) G protein signalling.

A shift from the inactive (dark-adapted) state to the active (G protein signalling) state is driven by absorption of a photon, and may be referred to as the ON response. A shift from the active (G protein signalling) state to the inactive (dark-adapted) state is also driven by absorption of a photon, and may be referred to as the OFF response. The relative efficiency with which photons drive ON vs OFF responses may vary according to wavelength. Put another way, a bistable opsin of the invention is one in which different wavelengths of light may be used to preferentially drive ON vs OFF responses or vice versa. Illumination with light only of a first wavelength will preferentially drive an ON response to the maximum available activity, and illumination with light only of a second wavelength will preferentially drive an OFF response to an inactive state or baseline level. A combination of light of the first and second wavelengths can be used to control the activity level of the bistable opsin. Herein by “sensitive” to a first or second wavelength of light means that the bistable opsin in a particular state responds most efficiently to said wavelength, or the photon at that wavelength is absorbed more efficiently than at another wavelength. Whilst other wavelengths of light may be absorbed by the bistable opsin in one state to drive the transition to the other state, the bistable opsin responds more efficiently to the first and second wavelengths of light for transition than any other wavelength of light. The bistable opsin in the inactive state may absorb more efficiently photons at a first wavelength than photons at any other wavelength. The bistable opsin in the active state may absorb more efficiently photons at a second wavelength than photons at any other wavelength.

A bistable opsin for use in the present invention may be mammalian, non-mammalian, vertebrate or non-vertebrate, plant, bacterial, or archeabacterial in origin. In an embodiment, the bistable opsin is a fish opsin, such as a lamprey opsin.

In an embodiment, the bistable opsin is a parapinopsin. A suitable parapinopsin may be lamprey parapinospin. The nucleic acid sequence encoding Lamprey Parapinopsin (Lethenteron camtschaticum or Lethenteron japonicum) is available at Genbank ID: AB116380.1. It may also be referred to as Lamprey PP or PPN, or LPPN.

Also included within the scope of the bistable opsins of the invention are variants or derivatives of the bistable opsin proteins. Variants include those sequences which share at least 70%, 75%, 80%, 85%, 90%, 95% or more sequence identity with a bistable opsin as described herein, such as the Lamprey Parapinopsin (Lethenteron camtschaticum or Lethenteron japonicum) available at Genbank ID: AB116380.1. Suitably, the variant and derivative sequences have the ability to react to light of different wavelengths to transition between active and inactive states, and mediate the G protein signalling pathway, as described herein. Sequence identity may be determined by any suitable method, for example as described herein. Derivatives of an opsin sequence as described herein include sequences comprising additions, deletions or substitutions of one or more residues of the native sequence. A variant or derivative may comprise nucleic acid sequence from other gene sequences, to provide a desired function or activity.

Also included are fragments of a bistable opsin, as described herein, which have the ability to react to light of at least two different wavelengths to transition between an active and inactive state and mediate G protein signalling pathway. A fragment may comprise a sequence comprising 70%, 75%, 80%, 85%, 90%, or 95% continuous amino acid residues of the native sequence. A fragment may comprise at least 50%, 60%, or 70% of the native sequence but no more than 70%, 75%, 80%, 85%, 90%, or 95% continuous amino acid residues of the native sequence.

In an embodiment, the bistable opsin is not naturally expressed in the cell to be transformed.

Light/Light Device

Herein, the two wavelengths at which the bistable opsin preferentially respond (or absorb photons) are referred to as the first and second wavelengths. Herein, the light of a first wavelength is the wavelength at which the bistable opsin most efficiently absorbs photons for transition from the inactive state to the active state. The first wavelength of light therefore most efficiently drives the ON response. Put another way, it may also be said that the bistable opsin is spectrally sensitive to light of the first wavelength. The light of a second wavelength is the wavelength at which the bistable opsin most efficiently absorbs photons for transition from the active state to the inactive state. The second wavelength of light therefore most efficiently drives the OFF response. Put another way, it may also be said that the bistable opsin is spectrally sensitive to light of the second wavelength of light.

By “sensitive” to a first or second wavelength of light means that the bistable opsin in a particular state responds most efficiently to said wavelength. Whilst other wavelengths of light may be absorbed to drive the transition to the other state, the bistable opsin responds more efficiently to the first and second wavelengths of light for transition than to any other wavelength of light.

Therefore, a bistable opsin of the present invention is spectrally sensitive to a first and second wavelength of light. By spectrally sensitive it is meant that the bistable opsin is preferentially sensitive to that wavelength, or responds most efficiently to that wavelength in terms of transition that to other wavelengths. However, it may transition between states upon absorption of other wavelengths of light, but with lower efficiency than at the first and second wavelengths. Efficiency of transition is the amount of bistable opsin which undergoes transition between active and inactive states at any given wavelength.

Herein, the term “wavelength” includes a range of wavelengths. Therefore, a first and a second wavelength may each independently define a single wavelength within the defined range, or a mixture of wavelengths within the defined range. Where a first and/or second wavelength of light each independently comprise a mixture of wavelengths, they may each independently comprise a maximal output at a preferred wavelength, for example one which the bistable opsin is particularly spectrally sensitive to.

In addition, a light source emitting light of the first and/or second wavelength may emit coloured light comprising wavelengths of light outside of the ranges of the first and/or second wavelengths. Suitable, such light sources will have a maximum output of light in the range of the first and/or second wavelength.

Thus, the light source may emit coloured light. The coloured light may suitably have a maximum output of light of a certain colour, for example blue or green.

Alternatively, the light emitted is monochromatic, and therefore all of a single wavelength. The wavelengths at which light most effectively drives the transition between active and inactive states in the bistable opsin preferably differ by at least 10 nm.

The ON response may be driven most efficiently by light in the wavelength of 380-450 nm, suitably 380-450 nm, more suitably 400 nm, more suitably 405 nm. The light may be monochromatic. Light in this wavelength is blue light. The bistable opsin may therefore be defined as being spectrally sensitive to these aforementioned wavelengths for transition between an inactive to an active state.

The OFF response may be driven by light in the wavelength of 450-650 nm, suitably 480-580 nm, more suitably 520 nm, more suitably 525 nm. The light may be monochromatic. Light in this wavelength is green light. The bistable opsin may therefore be defined as being spectrally sensitive to these aforementioned wavelengths for transition between an active to an inactive state.

Suitably, the device does not emit white light.

In certain embodiments, it is desirable to use a mixture of the two light colours, to regulate the amplitude of the response of the bistable opsin and the coupled G protein. Altering the ratio of light of a first and second wavelength emitted by the light source has been shown by the present inventors to change the degree of G protein signalling activation. Specifically, a higher proportion of blue light has been shown to drive a stronger G protein signalling response, and a higher proportion of green light has been shown to drive a stronger inactivation of G protein signalling response. The degree of the G protein response (or amplitude) can be directly controlled by altering the ratio of light of a first and second wavelength.

The ratio of light of a first and second wavelength may therefore be from 0:1 to 1:0, or anywhere in between. Specifically, the ratio may be 0.01:0.99 to 0.99:0.01. The amount of light of a first wavelength in the ratio may be 0.01 to 0.99, specifically 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, or 0.95 or any integer in between. For these amounts of light of a first wavelength light, the amount of light of a second wavelength combined therewith will be, respectively, 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, and 0.1.

The ratio of light may be the ratio of the energy of light at the specified wavelength.

The step of illuminating the cell comprises exposing the cell to a light stimulus, for a defined time period.

For vision applications, the cells may be illuminated may be one or more milli-seconds, or up to one or more hours, or indeed several hours, for any period of time where vision is required for example waking hours. A period of illumination may comprise one or more defined time periods or time intervals. A defined time period is any time period during which the illumination to which the cell is exposed has a constant wavelength or combination of wavelengths of light, including for example light of only a first wavelength, only a second wavelength, a combination of wavelengths within the first wavelength, a combination of wavelengths within the second wavelengths, and a combination of wavelengths of the first and second wavelengths. A defined time period or interval may be a millisecond, or more than one millisecond and any number of milliseconds up to and including 0.1 seconds, 0.2 seconds, 0.3 seconds, 0.4 seconds, 0.5 seconds, 0.6 seconds, 0.7 seconds, 0.8 seconds, 0.9 seconds or 1 second or more. A defined time period may be up to and including 2 seconds. The ratio of light of a first and second wavelength emitted during illumination may therefore be altered from any value from 0:1 to 1:0 (first wavelength:second wavelength) during the illumination period, any number of times.

Therefore, the ratio of light of the first wavelength: the second wavelength may be constantly shifting, for example every millisecond. A defined time period under a defined ratio of light of a first and second wavelength may comprise a period during which the substantial shift between states occurs, and may optionally comprise a period of equilibrium between states.

The ratio of light may be altered to achieve real time G protein regulation, for example real time control of the amplitude of a G protein response. In this way, altering the illumination and more suitably the ratio of light of the first and second wavelength may be used to alter in real time the visual response.

Any suitable light intensity may be used. Suitable light intensity may be from 9 and 18 log photons/cm2/s. The light intensity used may be selected depending upon the application of the invention. Where greater temporal resolution is required, a higher light intensity is more suitable. For example, for lamprey bistable opsin, a maximum light intensity of 14.31 log photons for 405 nm and 13.8 log photons for 525 nm may be suitable.

The light source may be provided in any suitable device. The device will suitably convert light intensity or other aspects of visual scene into their wavelength ratios. The type of device may depend on the application or condition to be treated. For an optogenetic aspect of the invention, the light source may be provided as eyewear, such as glasses, goggles or a headset. The device may have a dual function as a light source and a screen or visual display upon which an image is projected. For example, the device may be a virtual reality headset. In other applications, the device may be hand held, or may be provided in clothing, such as wearable technology. The device may be controlled by the user, and may be adapted for control remotely for example via a smartphone or similar device.

Target Cells

For optogenetic applications, a cell may be a retinal cell, preferably an inner retinal cell. A retinal cell may be a rod or cone cell, and/or may be a non-photoreceptor cell (i.e. a retinal cell which in its native form does not respond to light).

An inner retinal cell may be an ON-bipolar cell, an OFF-bipolar cell, a horizontal cell, a ganglion cell and/or an amacrine cell. Horizontal cells are inner retina cells, involved in signal processing and feedback to photoreceptor cells; bipolar cells are inner retinal cells and communicate between rods/cone cells and the amacrine and/or ganglion cells; amacrine cells are found in inner retina and allow communication between photoreceptor pathway and ganglion cells; ganglion cells are innermost retinal cells which pass signal from photoreceptor cells to the optic nerve.

A cell may include a population of cells, comprising one or more cell types be selected from the group consisting of rod cells, cone cells, ON-bipolar cells, OFF-bipolar cells, horizontal cells, ganglion cells, Muller cells and/or amacrine cells.

For non-retinal applications, a cell may be a brain cell, an endocrine cell, a nerve cell or may be a heart cell. A cell may be a stem cell, suitably a differentiated stem cell. A cell may include a population of cells. A population of cells may comprise a progeny of a stem cell, or one or more brain cell types, or one or more heart cell types, one or more endocrine cell types, or one or more nerve cell types.

A cell may be a prokaryotic or eukaryotic. It may be a bacterial cell such as E. coli, or may be a mammalian or non-mammalian cell, for example an insect cell, a yeast cell, a cell line or a cell free expression systems, for example for use in generating a vector or composition of the invention.

Reference to a cell herein includes progeny of the cell. Preferably, modifications to a cell according to the present invention also occur in succeeding generations of the transformed host cells. Progeny cells which may not be identical to the initial targeted cell but preferably will also exhibit expression of the non-native photosensitive protein.

A cell for use in the present invention may comprise the suitable G protein and signalling cascade. Therefore, a cell for use in the invention may naturally comprise Go and/or Gi. However, it is envisaged that there may be applications of the invention for use with cells which do not naturally comprise Go and/or Gi.

For vision applications, retinal cells are transformed with a nucleic acid sequence encoding a bistable opsin as defined herein, to compensate for degeneration of photoreceptor cells in the retina. The cells to which the nucleic acid sequence is targeted are cells of the retina which are suitably alive and capable of expressing a foreign nucleic acid sequence. Herein, a retinal cell is a cell of the retina, which is a nerve or neuron cell and is capable of becoming excited and transmitting an electrical signal. Preferably, a target retinal cell will be capable of generating an electrical signal and initiating the signalling cascade leading to transmission of signal to the optic nerve.

Thus, where a target retinal cell is an ON-bipolar cell, OFF-bipolar cell, horizontal cell, ganglion cell and/or amacrine cell of the retina, the expression of the nucleic acid encoding a bistable opsin may be referred to as ectopic or heterologous expression. Thus, the present invention includes within its scope a method of ectopically expressing a nucleic acid sequence encoding a bistable opsin protein in a non-photoreceptor cell. Such ectopic expression has the effect of providing photoreceptor function to a cell, by expression of a heterologous photosensitive protein therein. This serves to increase the photoreceptive capacity of the retina where degeneration is observed.

For non-vision applications, for example for the treatment of Parkinsons disease, Alzheimer's disease, schizophrenia or heart disease, the respective stem cells, brain, nerve, endocrine, or heart cells are transformed with a nucleic acid sequence encoding a bistable opsin as defined herein, to compensate for loss or partial loss of the Go protein signalling cascade. The cells to which the nucleic acid sequence is targeted are cells which are suitably alive and capable of expressing a foreign nucleic acid sequence. The cells may be native or non-native to the tissue to be treated. For example, stem cells may be transformed and introduced to a tissue to be treated. The cells may be encapsulated, for transplant to the tissue to be treated. A suitable cell may be one which is capable of becoming excited and transmitting an electrical signal. Preferably, a suitable cell will be capable of generating an electrical signal and initiating a signalling cascade leading to cell activation or deactivation.

Thus, where a target cell is a stem cell, heart, nerve, endocrine or brain cell, the expression of the nucleic acid encoding a bistable opsin may be referred to as ectopic or heterologous expression. Thus, the present invention includes within its scope a method of ectopically expressing a nucleic acid sequence encoding a bistable opsin protein in a non-photoreceptor cell. Such ectopic expression has the effect of providing a cell with a Go protein signalling cascade control mechanisms, in the form of the bistable opsin which naturally couples with the Go protein.

Heterologous

By “heterologous” is meant that the nucleic acid sequence is partly or entirely foreign (i.e., does not naturally occur in) to the transformed cell. Therefore, the nucleic acid encoding the bistable opsin is one which is not naturally expressed in the host cell which is to be transformed. Therefore, the cell to be transformed may not naturally express a bistable opsin; or may not naturally express the particular bistable opsin used in the method of the invention. Suitably, the bistable opsin may not be naturally present in the host cell.

Subjects

As used herein, the term “subject” refers to any mammal or non-mammal. Mammals include but are not limited to, humans, vertebrates such as rodents, non-human primates, cows, horses, dogs, cats, pigs, sheep, goats, giraffes, yaks, deer, camels, llamas, antelope, hares, and rabbits.

A subject may be one who has been diagnosed as suffering from, or being pre-disposed to, a retinal degenerative condition, for example selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability.

A subject may be one who has been diagnosed as suffering from, or being pre-disposed to, Parkinsons disease, Alzheimer's disease, schizophrenia or heart disease.

Nucleic Acid Sequence Encoding Bistable Opsin

A nucleic acid sequence encoding a bistable opsin may encode a full length native bistable opsin, or a fragment, variant or derivative of a bistable opsin. A nucleic acid sequence may be a DNA, RNA, cDNA, or PNA. It may be genomic, recombinant or synthetic. A nucleic acid sequence may be isolated or purified. It may be single stranded or double stranded. A nucleic acid sequence may be derived by cloning, for example using standard molecular cloning techniques including restriction digestion, ligation, gel electrophoresis, for example as described in Sambrook et al; Molecular Cloning: A laboratory manual, Cold Spring Harbour laboratory Press). A nucleic acid sequence may be isolated, for example using PCR technology. Such technology may employ primers based upon the sequence of the nucleic acid sequence to be amplified. By isolated is meant that the nucleic acid sequence is separated from any impurities and from other nucleic acid sequences and/or proteins which are naturally found associated with the nucleic acid sequence in its source. Therefore, it may be separated from flanking nucleic acid sequences, or from chromosomal material or sequence. Preferably, it will also be free of cellular material, culture medium, or other chemicals from a purification/production process. A nucleic acid sequence may be synthetic, for example produced by direct chemical synthesis e.g. using the phosphotriester method (Narang et al Meth Enzymol 68: 109-151 1979). A nucleic acid sequence may be provided as naked nucleic acid, or may be provided complexed with a protein or lipid. A nucleic acid for use in the invention may be altered to improve expression efficiency, or to alter desired characteristics of the response.

The sequence may be altered to improve expression efficiency (for example by truncating C-terminus or introducing targeting motifs), or to alter characteristics of the light response. (for example by removing or adding residues targeted by G-protein receptor kinases or arrestins as part of the signal termination process.

With the sequence information provided, the skilled person can use available cloning techniques to produce a nucleic acid sequence or vector suitable for transduction into a cell.

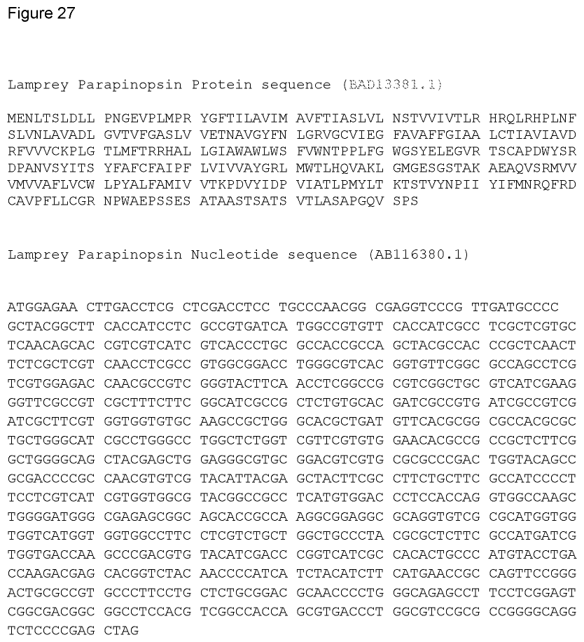

Nucleic acid sequences as described herein include fragments and derivatives of a native sequence. Preferably a fragment or derivative shares at least 70%, 75%, 80%, 85% or 90%, at least 91, 92, 93, 94, 95, 96, 97, 98, or at least 99% sequence identity with a native enzyme, over a length of 50%, 60%, 70%, 80%, 90%, or at least 95% of the length of a native sequence, for example LPPN. The sequence of LPPN is provided herein as FIG. 27. A nucleic acid sequence encoding a fragment of the bistable opsin may be a fragment of the full length nucleic acid sequence encoding the bistable opsin. It may comprise at least, or no more than, 50%, 60%, or 70% 75%, 80%, 85%, 90%, or 95% of the full length nucleic acid sequence. A nucleic acid encoding a variant or derivative may comprise additions, deletions or substitutions in the nucleic acid sequence, corresponding to the additions, deletions or substitutions in the amino acid sequence of the variant or derivative.

Sequence identity is determined by comparing the two aligned sequences over a pre-determined comparison window (which may be 50%, 60%, 70%, 80%, 90%, 95%, or 100% of the length of the reference nucleotide sequence or protein), and determining the number of positions at which identical residues occur. Typically, this is expressed as a percentage. The measurement of sequence identity of a nucleotide sequences is a method well known to those skilled in the art, using computer implemented mathematical algorithms such as ALIGN (Version 2.0), GAP, BESTFIT, BLAST (Altschul et al J. Mol. Biol. 215: 403 (1990)), FASTA and TFASTA (Wisconsin Genetic Software Package Version 8, available from Genetics Computer Group, Accelrys Inc. San Diego, Calif.), and CLUSTAL (Higgins et al, Gene 73: 237-244 (1998)), using default parameters.

Regulatory Sequences

One or more regulatory elements may be included in a nucleic acid sequence encoding a bistable opsin and/or in a vector comprising a nucleic acid sequence encoding a bistable opsin. Examples of regulatory elements include enhancers, promoters, transcription termination signals, polyadenylation sequences (for example SV40 late polyA), inverted terminal repeat, origin or replication, a tag, a nucleic acid restriction site, homologous recombination sites, Woodchuck Hepatitis Virus (WHP) Posttranscriptional Regulatory Element (WPRE), and a selection marker. Regulatory elements may be used to direct expression of the bistable opsin to the desired cells, for example ON bipolar cells. E which may be provided include

A promoter mediates expression of the nucleic acid sequence to which it is linked. A promoter may be constitutive or may be inducible. A promoter may direct ubiquitous expression in the inner retinal cells, or neurone specific expression. In the latter case, a promoter may direct cell type specific expression, for example to ON bipolar cells. Suitable promoters will be known to persons skilled in the art. For example, a suitable promoter may be selected from the group consisting of L7, thy-1, recoverin, calbindin, CMV (suitably human), GAD-67, chicken beta-actin, hSyn, Grm6, Grm6 enhancer-SV40 fusion protein. Targeting may be achieved using cell specific promoters, for example e.g. Grm6-SV40 for selective targeting of ON-bipolar cells. The Grm6 promoter is a fusion of 200-base pair enhancer sequence of the Grm6 gene encoding for ON-bipolar cell specific metabotropic glutamate receptor, mGluR6, and an SV40 eukaryotic promoter. Preferred sources of the Grm6 gene are mouse and human. Ubiquitous expression may be achieved using a pan-neuronal promoter, examples of which are known and available in the art. One such example is CAG. The CAG promoter is a fusion of CMV early enhancer and chicken β-actin promoter.

Suitably, a vector may comprise a nucleic acid sequence encoding a bistable opsin or a fragment or derivative thereof, under control of an ON bipolar cell specific promoter. The nucleic acid sequence encoding the bistable opsin may comprise a sequence tag and a polyadenylation sequence operably linked to the sequence encoding the bistable opsin. A Woodchuck Hepatitis Virus (WHP) Posttranscriptional Regulatory Element (WPRE) and an AAV ITR may also be included.

Vectors

Preferably, a nucleic acid sequence encoding a photosensitive protein is provided as a vector, preferably an expression vector. Preferably, it may be provided as a gene therapy vector, preferably which is suitable for transduction and expression in a target retinal cell. A vector may be viral or non-viral (e.g. a plasmid). Viral vectors include those derived from adenovirus, adenoassociated virus (AAV) including mutated forms, retrovirus, lentivirus, herpes virus, vaccinia virus, MMLV, GaLV, Simian Immune Deficiency Virus (SIV), HIV, pox virus, and SV40. A viral vector is preferably replication defective, although it is envisaged that it may be replication deficient, replication competent or conditional. A viral vector may typically persist in an extrachromosomal state without integrating into the genome of the target retinal cell. A preferred viral vector for introduction of a nucleic acid sequence encoding a photosensitive protein to a retinal target cell is an AAV vector, for example self-complementary adenoassociated virus (scAAV). Selective targeting may be achieved using a specific AAV serotype (AAV serotype 2 to AAV serotype 12) or a modified version of any of these serotypes including AAV 4YF (which includes 4 tyrosine to alanine mutations to achieve efficient viral transduction of retinal cells, in particular bipolar cells (Petra Silva et al 2011) and AAV 7m8 vectors. In aspects of the invention where the vector is provided by intra-vitreous administration, the vector may be one which has been modified such that it does not bind to one or more proteins of the ECM. For example, a preferred vector may comprise a modified heparin sulphate binding site, such that it has reduced or an inability to bind heperan sulphate, such as AAV 7m8 (Dalkara D et al Sci Transl Med 2013; 5:189ra76).

A viral vector may be modified to delete any non-essential sequences. For example, in AAV the virus may be modified to delete all or part of IX gene, E1a and/or E1b gene. For wild type AAV, replication is at extremely low efficiency, without the presence of helper virus, such as adenovirus. For recombinant adeno-associated virus, preferably the replication and capsid genes are provided in trans (in pRep/Cap plasmid), and only the 2 ITRs of AAV genome are left and packaged into a virion, while the adenovirus genes required are provided either provided by adenovirus or another plasmid. Similar modifications may be made to a lentiviral vector.

A viral vector has the ability to enter a cell. However, a non-viral vector such as plasmid may be complexed with an agent to facilitate its uptake by a target cell. Such agents include polycationic agents. Alternatively, a delivery system such as a liposome based delivery system may be used.

A vector for use in the present invention is preferably suitable for use in vivo or in vitro, and is preferably suitable for use in a human.

A vector will preferably comprise one or more regulatory sequences to direct expression of the nucleic acid sequence in a target retinal cell. A regulatory sequence may include a promoter operably linked to the nucleic acid sequence, an enhancer, a transcription termination signal, a polyadenylyation sequence, an origin of replication, a nucleic acid restriction site, and a homologous recombination site. A vector may also include a selectable marker, for example to determine expression of the vector in a growth system (for example a bacterial cell) or in a target retinal cell.

By “operably linked” means that the nucleic acid sequence is functionally associated with the sequence to which it is operably linked, such that they are linked in a manner such that they affect the expression or function of one another. For example, a nucleic acid sequence operably linked to a promoter will have an expression pattern influenced by the promoter.

A vector of the invention may be administered with or without an enzyme which degrades the extracellular matrix.

A nucleic acid sequence encoding a bistable opsin may be integrated into the genome of a target cell. It may be constitutively or transiently expressed. The cell may be a retinal cell, preferably an inner retinal cell such as an ON or OFF bipolar cell, horizontal cell, amacrine cell or ganglion cell. Most suitably the cell is a bipolar cell such as an ON bipolar cell.

Composition for Supply of Vector Encoding LPPN

The vector may be provided as a composition, for administration to a subject.

A composition may be a liquid or a solid, for example a powder, gel, or paste. Preferably, a composition is a liquid, preferably an injectable liquid. Suitable excipients will be known to persons skilled in the art.

Administration

Methods of treatment described herein may comprise administering an effective amount of vector to a subject for treatment. An effective amount or a therapeutically effective amount refers to an amount that is effective to relieve to some extent, or reduce the likelihood of onset of one or more symptoms of a disease or condition, and may include curing the disease or condition. Curing means that the symptoms of the disease or condition are eliminated. The administration of a nucleic acid vector comprising a nucleic acid encoding a human photoreceptor protein, the vector may be administered by injection or any other suitable means. For optogenetic applications, administration may be intra-ocular, suitably by intra-ocular injection. Suitably, administration to the eye may be by sub-retinal or intra-vitreous administration, suitably sub-retinal or intra-vitreous injection. For non-optogenetic applications, administration may be injection to the tissue or organ to be treated.

The vector is preferably provided as an injectable liquid. Preferably, the injectable liquid is provided as a capsule or syringe.

Kits

A kit of the invention may further comprise instructions for use, a dosage regimen, one or more fine needles, one or more syringes, and solvent. A kit may also comprise a light device as defined herein, and optionally a control mechanism for the light device. A software application may be provided for control of the device.

Retinal Conditions

A method of the invention may be used for the treatment of a retinal degenerative condition, for example a retinal dystrophy including a rod dystrophy, a rod-cone dystrophy, a cone-rod dystrophy, a cone dystrophy and a macular dystrophy; another form of retinal or macular degeneration, an ischaemic condition, uveitis, edema, and any other disease resulting from loss of photoreceptor ability.

The methods of the invention can be used to provide photoreceptor function to a cell which previously did not have photoreceptor ability or whose photoreceptor ability has degenerated, wholly or partially. Provision of a bistable opsin as described herein to a cell enables the cell to become photo-receptive upon expression thereof. Such a cell may be referred to herein as a transformed cell, because it comprises therein non-native nucleic acid.

A transformed retinal cell exhibits some or all of the photoreceptor ability of a native photoreceptive cell. Preferably, a transformed cell exhibits at least the same or substantially the same photoreceptive ability of a native retinal photoreceptor cell. Preferably, a transformed cell exhibits higher photoreceptive ability than a diseased or degenerating native retinal photoreceptor cell. Therefore, a transformed cell will preferably have increased photoreceptor compared to a degenerated or diseased cell from the same source, maintained under the same conditions, without treatment. A transformed cell can be distinguished from a native cell by the presence therein of exogenous nucleic acid.

By augmenting photoreceptor function is meant increasing photoreceptor function of the retina, either by increasing the function in photoreceptor cells such as rod or cone cells, and/or by providing photoreceptor function to a cell. Thus, the retina will have an increased ability to receive light signals and transmit such signals compared to a retina which has not been treated with method as described herein. The increase may be by any amount, preferably to wild type levels.

By restoring vision in a subject is meant that the subject shows improved vision compared to prior to treatment, for example using vision tests as described herein. Restoring includes any degree in improvement, including full restoration of vision to perfect or near perfect vision.

By treating disease is meant administration of a nucleic acid as described herein to ameliorate or alleviate of one or more symptoms of a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability, Parkinsons disease, Alzheimer's disease, schizophrenia and heart disease. Amelioration or alleviation may result in an improvement of peripheral or central vision, and/or day or night vision; improvement of cognitive and motor functions; or improvement in vagal heart rhythm.

Methods of the invention comprise introducing into the vitreal cavity of an eye a nucleic acid sequence encoding a photosensitive protein. Preferably, the method comprises contacting a cell with a nucleic acid sequence encoding a photosensitive protein. Preferably, a cell is a retinal cell, preferably an ON-bipolar cell, an OFF-bipolar cell, a horizontal cell, a ganglion cell and/or an amacrine cell.

Preferably, a method of the invention comprises targeting a nucleic acid sequence encoding a photosensitive protein to the retina of an eye, preferably to a non-photoreceptive cell of the retina, preferably to an ON-bipolar cell, an OFF-bipolar cell, a horizontal cell, a ganglion cell and/or an amacrine cell. Thus, by contacting a cell includes transfection and/or transduction of a cell.

Where an enzyme is administered, the enzyme does not need to be internalised into a retinal cell, but may remain in the vitreal cavity or in the retina, where it degrades extracellular matrix proteins to improve access of the nucleic acid sequence to the retinal cells.

A method of the invention is preferably performed in vivo.

Dosage

Each dose may comprise an effective amount of a nucleic acid sequence. An effective dose of a nucleic acid sequence may range from 1×10⁹ to 1×10¹⁴ or 7.5×10¹⁵, preferably 1×10¹¹ to 7.5×10¹³ nucleic acid sequences per treatment regimen (e.g. number of vectors or virus particles).

Diagnosis

A method of the invention may comprise a step of diagnosing a subject for a retinal degenerative condition, for example a retinal dystrophy including a rod dystrophy, a rod-cone dystrophy, a cone-rod dystrophy, a cone dystrophy, a macular dystrophy; another forms of retinal or macular degeneration, an ischaemic conditions, uveitis and any other disease resulting from loss of photoreceptor ability. A diagnostic step may comprise a visual test, for example a pupillary light reflex (PLR) test, visual acuity test (LogMAR), clinical diagnostic tests for example biomicroscopy/slit-lamp ocular/retinal clinical examination; colour vision testing, visual field testing, contrast/full field sensitivity; electrodiagnostic tests including for example EGGs, VEPs; imaging, retinal fundus photography, OCT, and adaptive optics scanning laser ophthalmoscope (AOSLO). Other suitable tests will be known to persons skilled in the art.

A method of the invention may also comprise the step of diagnosing a subject for Parkinsons disease, Alzheimer's disease, schizophrenia or heart disease.

Pre-Administration

A method of the invention may comprise a step of dilating the pupil of an eye to be treated, for example by application of mydriatic, for example tropicamide and/or phenylephrine and/or cyclopentolate. A method of the invention may comprise a step of accessing the retina, for example by surgery.

A method of the invention may comprise accessing the brain for injection or surgery.

A method of the invention may comprise accessing the heart, for example by injection.

Monitoring

A method of the invention may comprise monitoring the vision of a subject who has been treated for any improvement in vision. Improvements in vision may be any one or more of the following: increased pupillary light reflex (PLR), increased contrast sensitivity, increased resolution of low or high frequency flicker, improved ability to navigate an environment with visual cues, and increased detection of moving images. In addition, increased light induced locomotor activity may be improved in animals such as mice. An improvement in vision may be an ability to respond to or detect light at 10¹⁵-10¹³ photon/cm²/s corneal irradiance. An improvement in vision may comprise an ON-sustained, ON-transient, OFF-excitatory, OFF-inhibitory or ON-OFF response for a visual neuron or group of neurons. Preferably, monitoring improvement may comprise a method of quantifying the subject's subjective visual experience or an objective measure of light response, for example a pupillary light reflex (PLR) test, LogMAR visual acuity, clinical examination slit-lamp biomicroscopy; colour vision testing, visual field testing, contrast/full field sensitivity; electrodiagnostics—ERGs, VEPs; imaging: retinal fundus photography, OCT, adaptive optics scanning laser ophthalmoscope (AOSLO), or maze navigation tasks.

A method of the invention may comprise monitoring the cognitive and motor functions of a subject who has been treated, for any improvement thereof.

A method of the invention may comprise monitoring heart function of a subject who has been treated, for any improvement thereof.

A subject may be monitored every 6, 8, 10, 12 or 24 hours, or every 2, 3, 4, 5 days. This may be repeated after 1, 2, 3, 4, 5, 6 months or a year or more.

Herein reference to “a” or “an” includes within its scope both the singular, and the plural, i.e. one or more.

Unless stated otherwise, the features and embodiments of each aspect applies to the other aspects of the invention, mutatis mutandis.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The present invention includes any suitable combinations of the features and suitable features described herein. The present invention includes combinations of features selected from lists of preferred features, for example where embodiments of a first feature are described as A, B, C, and embodiments of a second feature are described as AA, BB, CC, the present invention includes any combination of features including AAA, ABB, ACC, BBB, BAA, etc.

The reader's attention is directed to all papers and documents which are filed concurrently with or previous to this specification in connection with this application and which are open to public inspection with this specification, and the contents of all such papers and documents are incorporated herein by reference.

### EXAMPLES

Cell Based Data

Cell Culture and Transfections

Hek293T cells (ATCC) were incubated at 37° C. (5% CO₂) in culture media (Dulbecco's modified Eagle's medium with 4500 mg/L glucose, L-glutamine, sodium pyruvate and sodium bicarbonate from Sigma) with penicillin (100 U/ml), streptomycin (100 μ/ml) and 10% fetal bovine serum (FBS)

For transfections, cell were seeded into 12-well plates at a density of 250 000 cells/well in antibiotic-free culture medium. After 48 hrs, cells were transiently transfected using Lipofectamine 2000 (Thermo Fisher) according to manufacturer's instructions. For all transfections, total amount of DNA was normalised between conditions using empty plasmid vector.

For BRET G protein activation assay, each well of 12-well plate was transfected with following:

100 ng sVβ1, 100 ng sVγ2, 100 ng mGRK3-nLuc, 200 ng Gao, 500 ng opsin or empty plasmid vector. All BRET assay plasmids were obtained from Kiril Martemyanov & Ikuo Masuho (Scripps Research Institute). The lamprey parapinopsin plasmid was supplied by Akihisa Terakita (Osaka City University).

As previously described in Masuho et al ((2015 Sci. Signal., 8(405): ra123. DOI: 10.1126/scisignal.aab4068) the 2:1 ratio of Gαo_{Aser} to sV β1, sVγ2 and mGRK3-nLuc is important for obtaining maximal response amplitude and low basal BRET ratio. All subsequent steps were conducted under dim red light. After addition of transfection reagent and DNA cells were incubated for 4-6 hours at 37° C., then resuspended in 2 ml of culture media containing 10 μM 9-cis retinal. 75 μl of cell suspension was added to each well of a white-walled clear-bottomed 96-well plate (Greiner Bio-One) and left overnight before performing BRET G protein activation assay.

BRET G Protein Activation Assay

Approximately 2 hours before beginning BRET G protein activation assay, culture media was removed from cells and replaced with 50 μl imaging media (L-15 media without Phenol Red containing L-glutamine (Gibco), 1% FBS, penicillin (100 U/ml) with 10 μM 9-cis retinal). Cells were then left to incubate at room temperature in dark for up to 2 hours.

Under dim red light, NanoGlo Live Cell substrate (Furimazine derivative, Promega) was diluted 1:40 in PBS. Then 12.5 ul of dilute NanoGlo substrate solution was added to each well of 96-well plate (to provide final dilution of 1:200 of NanoGlo substrate), for up 6 wells at a time, and incubated for 10 mins before commencing assay to allow luminescence to peak.

BRET measurements were conducted using a FluoStar Optima microplate reader (BMG Labtech). As this plate reader has a single photomultiplier tube, light emitted by fluorescent Venus and bioluminescent Nanoluc were measured sequentially using 535 nm (30 nm FWHM with gain set to 4095) and 470 nm (30 nm FWHM with gain set to 3600) emission filters. A 0.68 s recording interval was used for each filter, with a total cycle time of 2 s.

To avoid delays associated with pausing recording, removing plate from reader for light exposure and returning plate to reader to resume recording, we adapted the plate reader bottom optic to allow us to deliver light to individual wells inside the plate reader. A custom-printed 3D coupler was used to connect the bottom optic with the liquid light guide of a pE-4000 light source (CoolLED). Combined with clear-bottomed 96-well plates, this allowed us to provide a light stimulus below cells. To avoid bleaching the PMT during light stimulus, a motorized shutter was built to protect the PMT while light was on. The activity of this shutter was synced to the light source using an Arduino microcontroller. To avoid neighbouring wells being exposed to light, each recorded well was surrounded by empty wells and the order of wells measured was counterbalanced.

Each BRET recording consisted of 2 kinetic windows. The first consisted of 5 cycles of baseline measurement (total duration 10 s) and the second protocol began after a short 3-4 s delay (during which cells were exposed to the delayed light pulse) for up 45 cycles (total duration 90 s). Plate reader software (Optima in script mode) was used to send an executable file to the Arduino controlling the shutter and light source, so that a 1 s light pulse was triggered to occur immediately before recording resumed. For experiments involving repeated light exposures (for example alternating blue and green lights), sequential recordings of the same well were performed (each with same baseline and response durations as outlined above).

A range of different light stimuli were used (all 1 s in duration)—detailed in FIG. 6 and Table 1. The spectral power distribution of the stimuli was measured using a SpectroCAL spectroradiometer (Cambridge Research Systems).

Results

A BRET assay was used which allows direct measurement of the amount of G-protein activated by opsin in real time (Masuho et al., 2015). It is shown in cartoon form in FIG. 4.

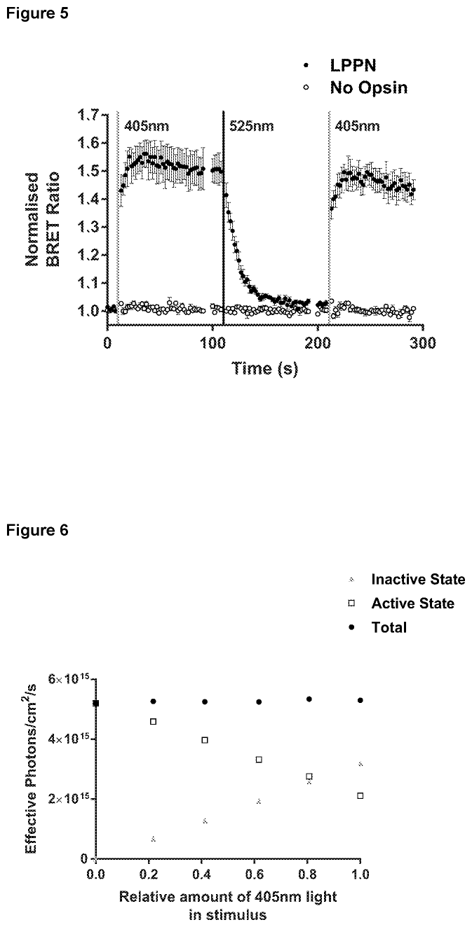

The inventors asked whether Lamprey parapinopsin could signal via GalphaO and if ON and OFF responses could be observed with the Go BRET assay. A blue monochromatic 405 nm was used as the ON stimulus and green monochromatic 525 nm as the OFF light stimulus. It was found that the blue ON stimulus was able to rapidly drive strong Go activation (FIG. 5). This appeared to be sustained at a high level, until the green OFF stimulus was applied, when Go activity returned to baseline. A subsequent blue ON stimulus was able to drive a similarly high level of Go activity, with no sign of bleaching. The OFF response is also considerably faster than that of Rod opsin (see FIG. 4b), but has a comparable response amplitude.

It was next asked whether the amplitude of the BRET light response (the amount of G protein activation) could be controlled by manipulating the relative amount of blue or green light in our stimulus. Based on the spectral sensitivity of the active and inactive states, a set of stimuli (FIG. 6 and Table 1) was generated that all had the same number of total effective photons, but varied the ratio of how much they would excite the inactive or active states of lamprey parapinopsin (driving ON or OFF responses respectively). Because the active and inactive states exist in equilibrium, these mixed stimuli should result in a clearly defined level of BRET light response.

TABLE 1
Stimuli created by mixing different amount of monochromatic blue (405 nm) and
green (525 nm) light. The spectral sensitivity of the inactive and active state of lamprey
parapinopsin is known, and enables the creation of stimuli that have known ratio of
effective photons for active and inactive state.

| Relative | Effective | Effective | | Relative | |
|---|---|---|---|---|---|
| amount of | photons/ | photons/ | | excitation | |
| Blue | cm²/s | cm²/s | | of | Relative |
| (405 nm) in | for Inactive | for Active | Total effective | “inactive | excitation of |
| stimulus | State | state | Photons/cm²/s | state” | “active state” |
| 0 | 7.21E+11 | 5.19E+13 | 5.19E+13 | 0.00 | 1 |
| 0.21 | 6.71E+12 | 4.58E+13 | 5.26E+13 | 0.13 | 9.97 |
| 0.41 | 1.28E+13 | 3.96E+13 | 5.24E+13 | 0.24 | 9.75 |
| 0.61 | 1.93E+13 | 3.31E+13 | 5.24E+13 | 0.37 | 9.63 |
| 0.80 | 2.58E+13 | 2.75E+13 | 5.33E+13 | 0.48 | 0.51 |
| 1 | 3.19E+13 | 2.10E+13 | 5.29E+13 | 0.60 | 9.39 |

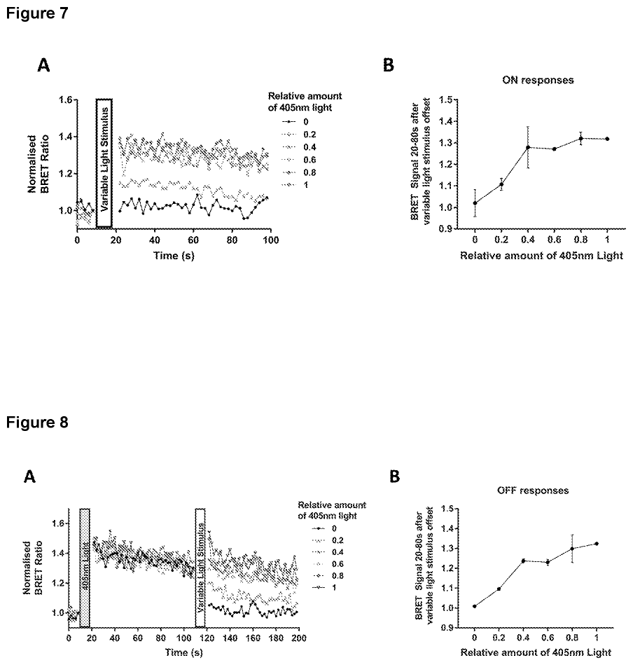

It was found that for dark adapted cells (LPPN mostly in the inactive state), increasing the relative amount of blue in the stimulus resulted in an increase in BRET light ON response (FIG. 7). This is consistent with increasing excitation of the inactive state (ie: driving more ON response) by blue light.

It was also asked whether similar results would be observed for the OFF responses of light-exposed cells (mostly in the active state). It was found that decreasing the relative amount of blue (and the corresponding increase in relative amount of green) in the stimulus resulted in larger OFF responses (larger decreases in BRET light signal, FIG. 8). This is consistent with increasing excitation of the active state by green light.

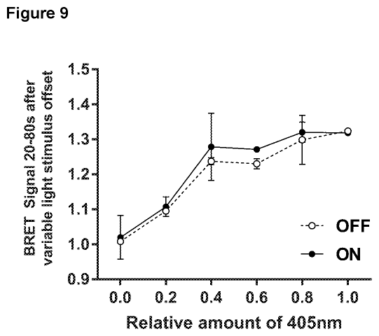

It was found that it is possible to drive the same amount of G protein activation from either a dark adapted ON response or light-exposed OFF response (FIG. 9). This suggests the ability to precisely control the level of G protein activation using wavelength ratio, independent of previous light exposure or current signalling state.

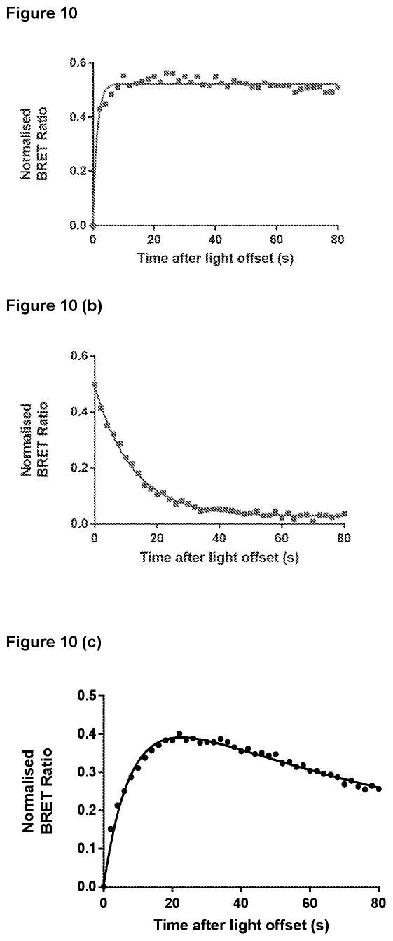

Using the live cell BRET assay, the kinetics of LPPN were found to be much faster than other Gi/o-coupled opsins, such as Rod opsin. For example, G protein activation of LPPN had a half life of 1.35 s (FIG. 10a) and G protein deactivation half-life of 8.46 s (FIG. 10b). This deactivation may be even faster (comparable to activation), with slower speed likely due to delay associated with BRET reporter (dissociation of luciferase-tagged GRK3 fragment from fluorescent-tagged BY dimer). For comparison, Rod opsin has a half-life of activation of 6 s and a deactivation of 84 s, when measured using the same assay (FIG. 10c).

This data shows that it is possible to achieve quantitative and near real-time activity of G-protein signalling by expressing a bistable opsin that has a substantial (>10 nm) difference in the spectral sensitivity of its two pigment states, in a cell type of interest and illuminating that cell with a lighting device in which the spectral power distribution could be modulated. At its simplest, the lighting device could have independently controllable coloured lights.

Thus, the data provides the basis for introduction of lamprey parapinopsin to target tissue, such as bipolar cells, which could then be controlled using a lighting device that would be able to deliver a precise amount of blue and green light. Such a system would be suitable for controlling Gi/Go pathways.

In the case of vision restoration, the target cells could be any inner retinal neuron (for example, bipolar cells, amacrine cells, horizontal cells, ganglion cells). Suitably, the target cells may be bipolar cells, most suitably ON bipolar cells, in which parapinopsins ability to interact with Go would recapitulate the native G-protein signalling event in vision. The data herein demonstrates that it is possible to quantitatively control the degree of G-protein activity by mixing two coloured lights. Such expression can be coupled with a visual display (e.g. goggles or virtual reality device) to translate patterns in the outside world into variations in the spectral power distribution of emitted light. Differences in brightness would be translated into variations in spectral power distribution (colour) and thus to patterns of G-protein activity in the retina. An online control of colour and also intensity could allow each patient to adjust the device to provide optimal vision.

In Vivo Data

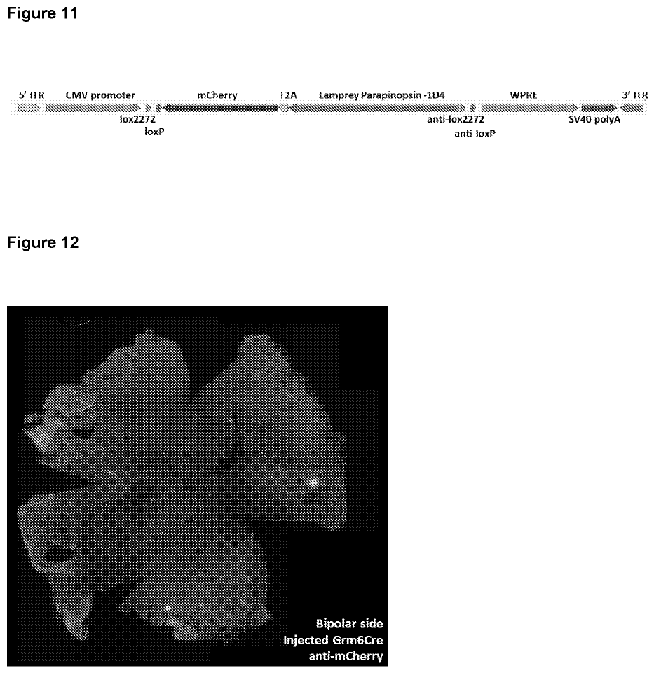

The nucleic acid vector used in the methodology described herein is an adeno-associated virus (AAV2 Quad-YF) containing the lamprey parapinopsin (LPPN) transgene (with C-terminal 1D4 tag). The lamprey parapinopsin is co-expressed with a fluorescent mCherry reporter (FIG. 11). The virus is floxed and will only express in cells expressing Cre-recombinase.

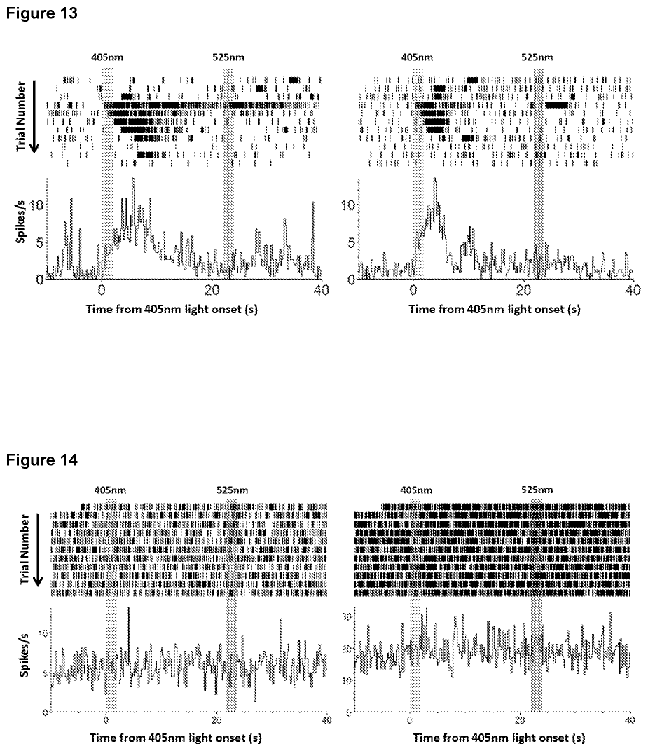

We have performed intravitreal injections of the LPPN AAV in retinally degenerate Grm6Cre rd1 mice, which express Cre-recombinase under control of the Grm6 promoter exclusively in ON bipolar cells. Immunofluorescence staining for mCherry reveals the virus successfully transduced the retina and is expressed at a relatively high density with good distribution across the retina (FIG. 12). Multi-electrode array recording on retinal explants from injected and uninjected mice were performed and responses to 2 s flashes of 405 nm and 525 nm light separated by 20 s of dark, over 10 trials were measured. It was observed that the retina expressing Lamprey parapinopsin exhibited transient responses to 405 nm, but not 525 nm flashes of light (FIG. 13). No responses from uninjected negative control retinas from rd1 mice were observed (FIG. 14), suggesting the light responses observed in the retinas expressing lamprey parapinopsin were not driven by melanopsin and/or any residual rod and cone photoreceptors that may have survived retinal degeneration.

This finding demonstrates that the Lamprey parapinopsin can be successfully expressed in the mouse retina and is functional. It can couple to the bipolar cell signalling pathway and makes these cells directly photosensitive. Evidence that lamprey parapinopsin demonstrates responses to the short, but not long, wavelength of light is also positive. This is consistent with the two known spectral states of lamprey parapinopsin—where short wavelength light leads to activation of the opsin and longer wavelength light leads to deactivation.

Intravitreal injections of the LPPN AAV as described above in retinally degenerate L7^{Cre/+} rd1 mice were performed. These mice have restricted expression of Cre recombinase under control of the L7 promoter in rod bipolar cells and a small subset of ganglion cells (Ivanova et al (2010) Neuroscience 65(1) 233-243)

(https://doi.org/10.1016/j.neuroscience.2009.10.021)

Multi-electrode array recordings were performed on retinal explants from lamprey parapinopsin AAV injected mice and responses to two wavelengths were measured: 405 nm (which predominantly drives transition from inactive to active state) and 525 nm (which drives transition from active to inactive state) or a 50:50 mix of the two. Herein these are referred to as blue, green and mixed light stimuli, respectively.

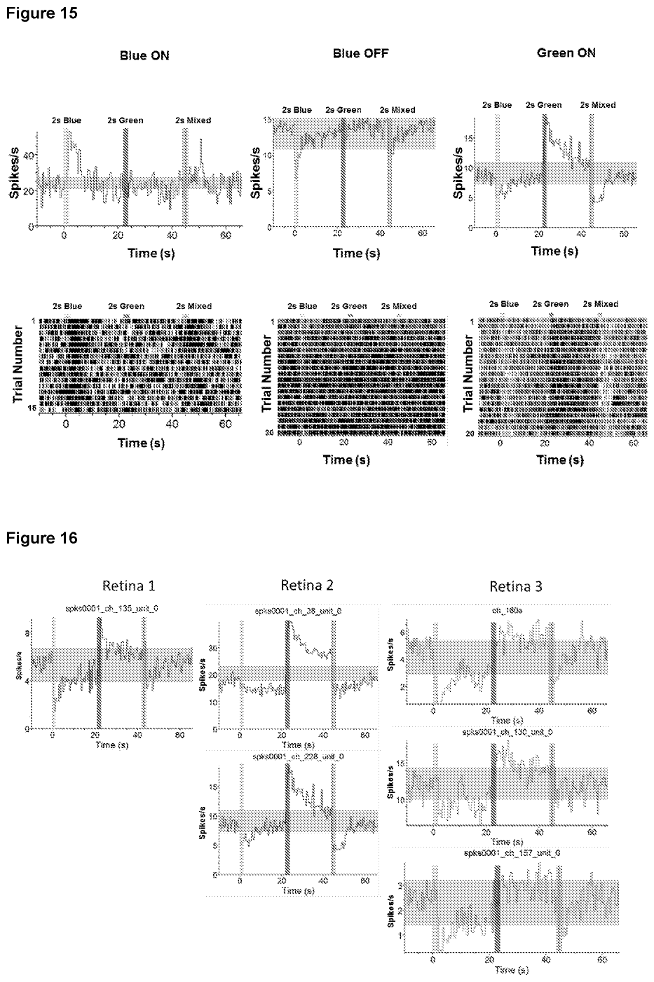

It was found that L7^{Cre/+} retinas expressing lamprey parapinopsin exhibited a range of responses to 2 s flashes of these light stimuli (FIG. 15). Some MEA channels recorded from cells excited by blue and mixed light, which did not appear to respond to green light (referred to as “Blue ON”). Channels were found where activity was inhibited by blue and mixed light, but did not respond to the green light (referred to as “Blue OFF”). Surprisingly, some channels showed inhibition to 405 nm light and excitation to 525 nm light (referred to as “Green ON”). Notably, light responses were highly sustained across multiple trials, which is consistent with lamprey parapinopsin being a non-bleaching pigment.

FIG. 15 shows examples of different types of light responsive channels from multi-electrode array recordings of rd1 mouse retinas expressing lamprey parapinopsin in rod bipolar cells and retinal ganglion cells. These include excitation by blue light (“Blue ON, left), inhibition by blue light (Blue OFF, middle) and excitation by green light but also inhibition by blue light (Green ON, right). Top panel shows perievent spike firing rate histograms averaged across 16-20 trials (bin size=500 ms). Timing of 100% 405 nm (“blue”), 100% 525 nm (“green”) and 50% 525 nm 50% 405 nm light (“mixed”) 2 s light flashes at 0 s, 22 s and 44 s are shown with shaded vertical bars. Horizontal shaded bar shows the 95% confidence interval for spike firing rate. Bottom panel shows raster plots of spike firing across multiple trials. Each row represents a different trial, with thicker width bars indicating increased spike firing rate. Timing of light stimuli is shown by boxes above raster plots. While some Blue ON and Blue OFF responses appeared to be relatively transient, it was found that responses in multiple Green ON units were sustained after the stimulus had ended, continuing until the next flash was presented. This is consistent with the previous findings in Hek293T cells, where blue light causes sustained increase in G protein activity until presentation of green light, which caused sustained decrease in G protein activity. As a bistable opsin, the active state of lamprey parapinopsin is thermally stable. This means it is able to continue signalling, without releasing its chromophore, until subsequent illumination with a different wavelength of light.

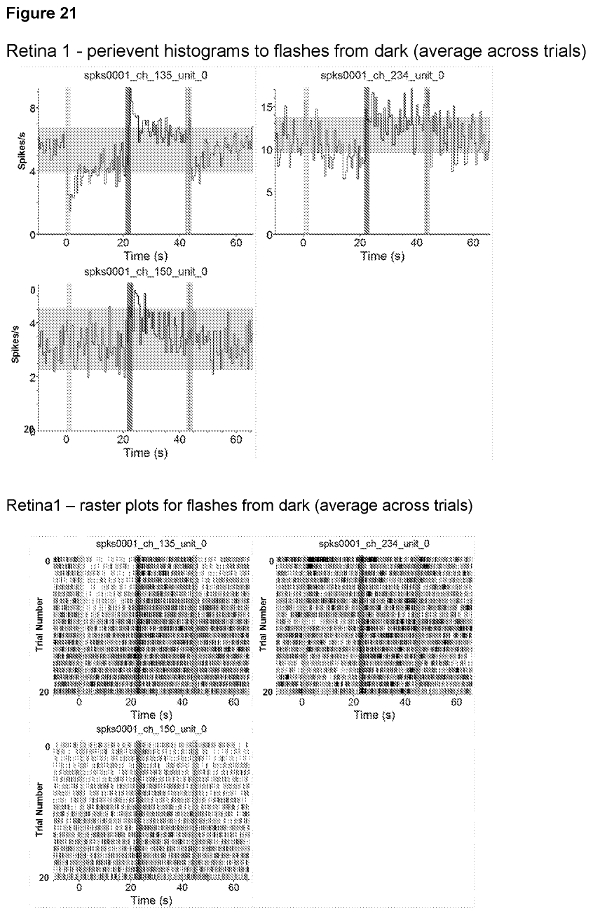

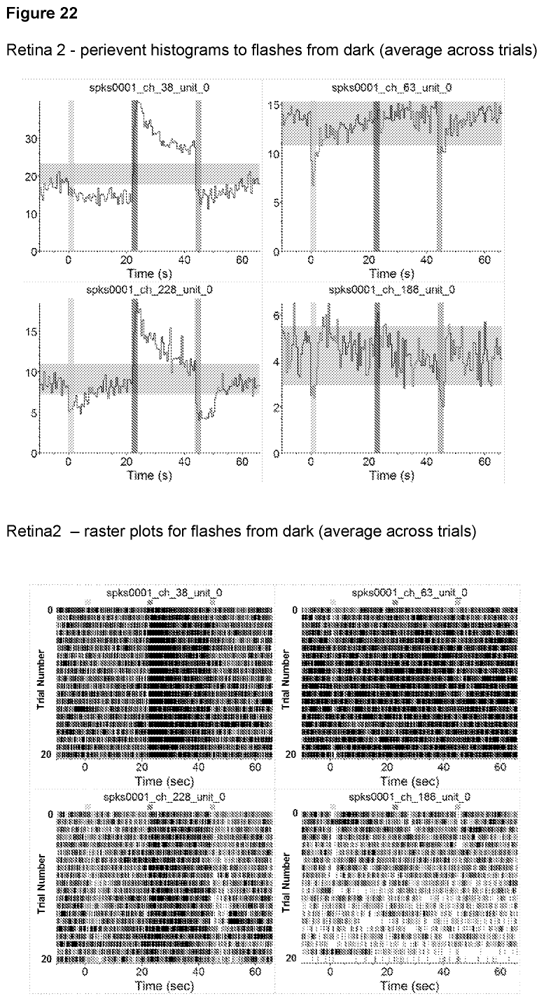

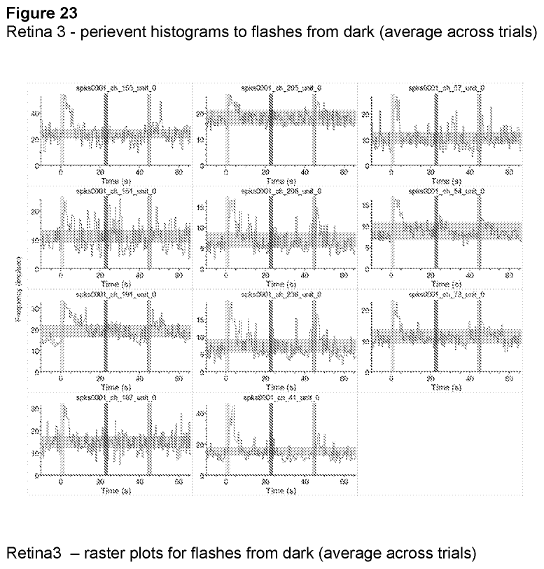

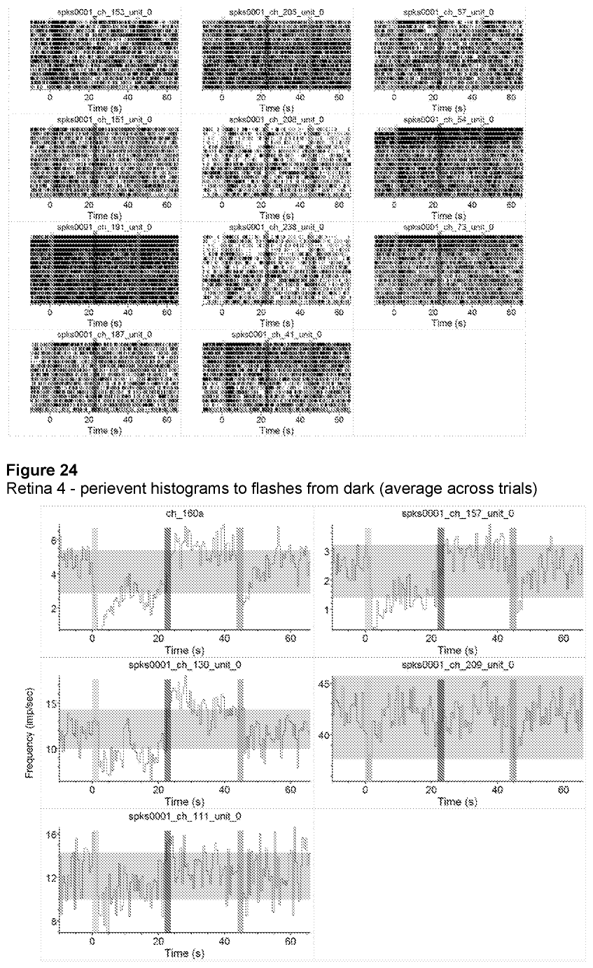

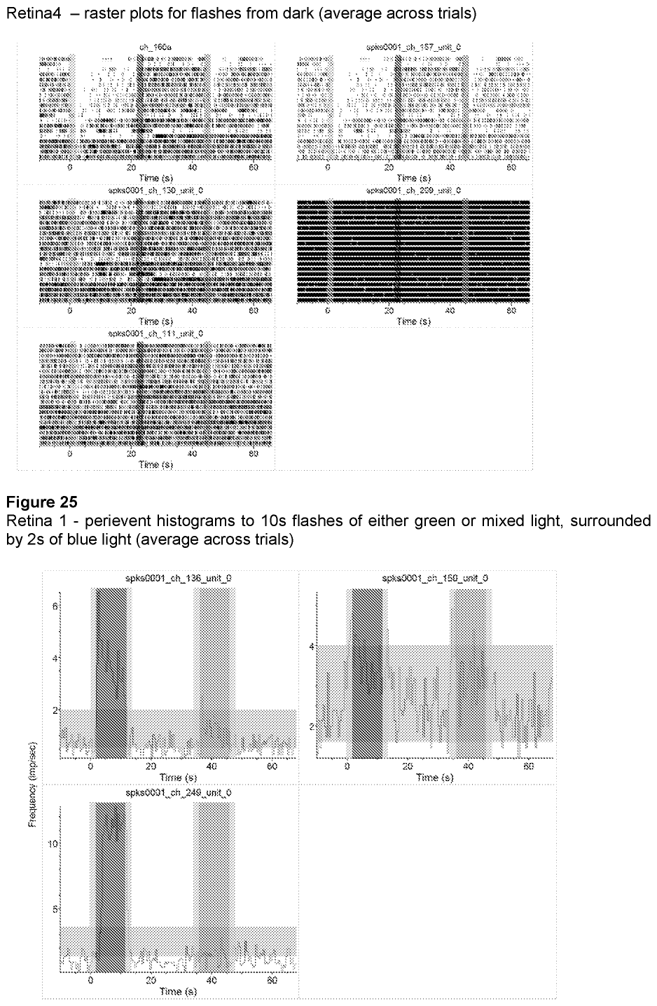

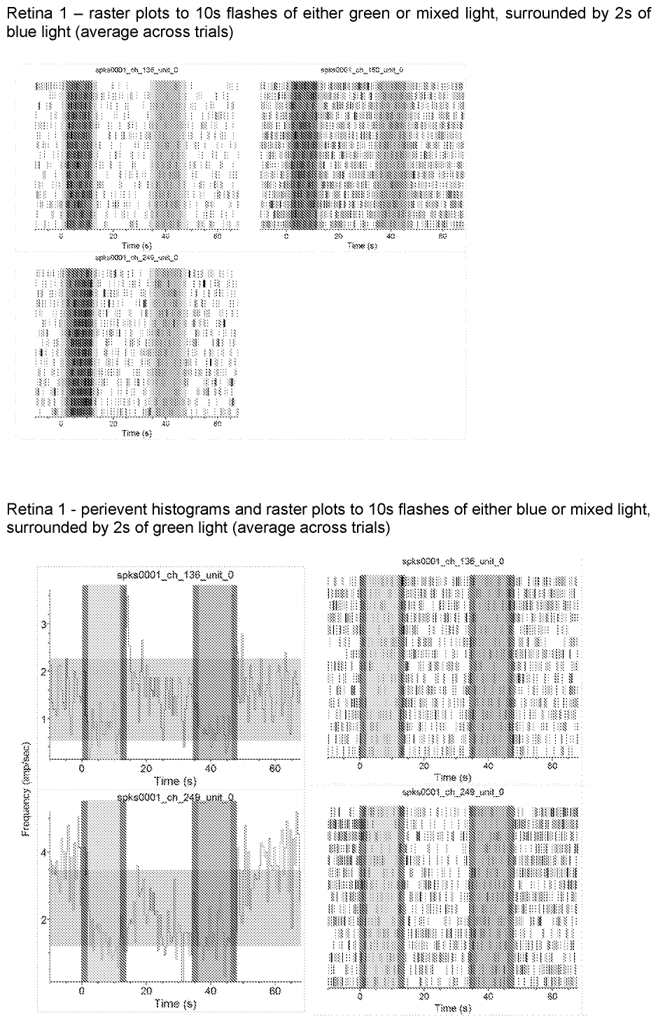

All the types of light responses observed are consistent with the different impact blue and green light has on lamprey parapinopsin activity. Of particular interest is the observation that it is possible to drive opposite signs of activity in the Green ON cells using different wavelengths. These Green ON responses were observed across multiple different retinas (FIG. 21, FIG. 22 and FIG. 24), suggesting they are a robust property of retinal cells transduced with lamprey parapinopsin. In FIG. 16 example channels with Green ON light responses are shown for 3 different retinas (grouped into columns). Perievent spike firing rate histograms show average across 16-20 trials (bin size=500 ms). Timing of 2 s 100% 405 nm (“blue”—light grey), 100% 525 nm (“green”—dark grey) and 50% 525 nm 50% 405 nm light (“mixed”—medium grey) light flashes at 0 s, 22 s and 44 s, respectively, are shown with shaded vertical bars. Horizontal shaded bar shows the 95% confidence interval for spike firing rate.

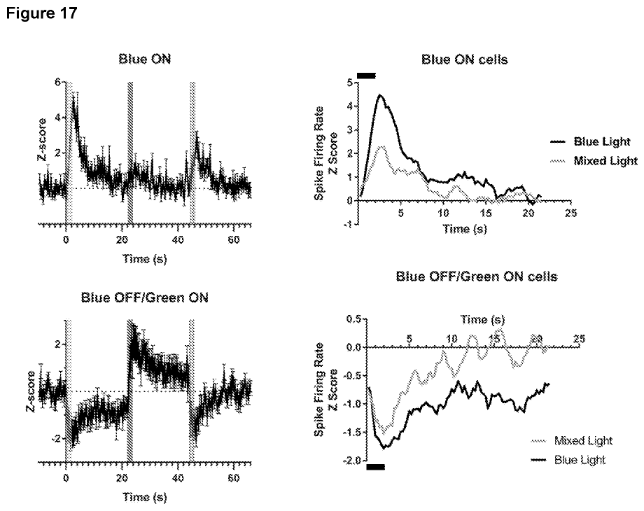

One of the previous important findings in the in vitro data was that the level of G protein activity could be precisely controlled using different ratios of blue and green light. Excitingly, it was possible to replicate this finding in vivo. Regardless of sign, light responses to the mixed light stimulus were attenuated compared to the blue light stimulus. In FIG. 17, the top panel shows light responses where blue and mixed light cause excitation (Blue ON, n=11 channels from 1 retina. For responses from individual channels see FIG. 23). Bottom panel shows inhibitory light responses to blue and mixed light (Blue OFF/Green ON, n=13 channels from 3 retinas. For responses from individual channels see FIG. 21, FIG. 22 and FIG. 24). Spike data was sorted into 250 ms bins. A z-score (number of standard deviations from baseline) was calculated by subtracting the baseline mean, then dividing each bin by baseline standard deviation. In the left panel, average responses are shown to 2 s flashes of either 100% 405 nm (blue light) and 50% 405 nm 50% 525 nm (mixed light) at 0 s, 22 s and 44 s, respectively (timing shown with shaded vertical bars). Note spike firing rate relative to baseline is higher in Blue ON and lower in Blue OFF/Green ON units after blue compared to mixed light flash. Error bars show standard error of mean. Right panel shows direct comparison of activity after either blue or mixed light. Z-score data was smoothed using a 5 bin rolling average. Stimulus timing and duration is shown by black bar above or below each graph.

These stimuli have the same total effective photons for lamprey parapinopsin and only differ in their spectral composition, indicating that mixing these two wavelengths is a potentially useful method for controlling the activity of lamprey parapinopsin.

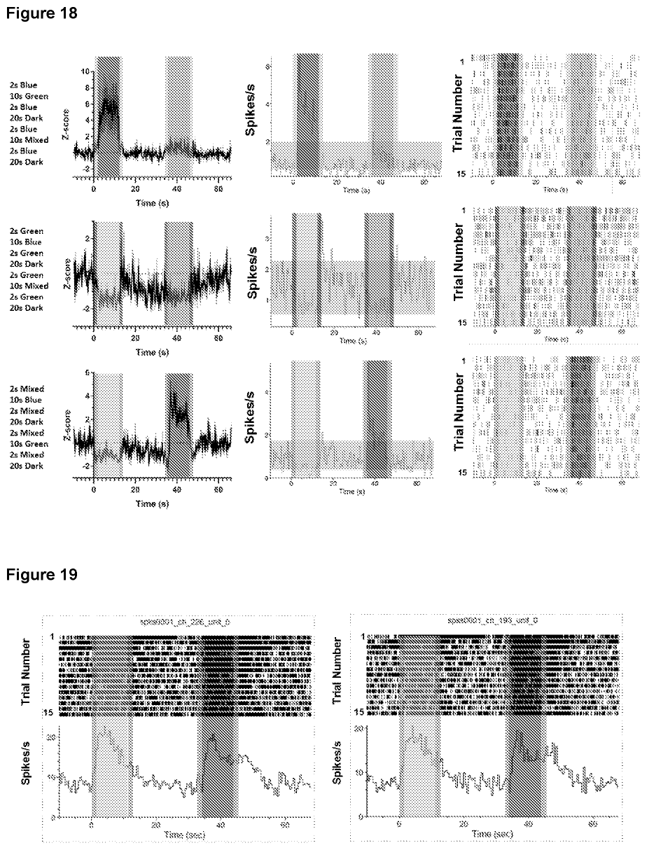

It was also tested whether sequential exposure to different wavelengths of light could be used to control activity of transduced retinal cells. To do this, sequential light stimuli of different wavelengths was used without dark adapting between each wavelength. Different sequences of short (2 s) and long (10 s) light exposures were used. In FIG. 18, the Left panel shows average time-course for light responses to different light stimuli (n=3-4 units from 2 retinas). Perievent spike firing rate histograms were calculated across 16-20 trials (bin size=250 ms). A z-score (number of standard deviations from baseline) was then calculated by subtracting the baseline mean, then dividing each bin by baseline standard deviation. Error bars show standard error of the mean. Middle panel shows perievent spike firing rate histograms for the same example Green ON unit across different light conditions (bin size=500 ms). Horizontal shaded bar shows 95% confidence interval. Right panel shows raster plots for the same example Green ON unit across different light conditions. For all graphs, vertical shaded bars show timing of light stimuli, either 100% 405 nm (“blue”—light grey), 100% 525 nm (“green”—dark grey) and 50% 525 nm 50% 405 nm light (“mixed”—medium grey) for either 2 s or 10 s.

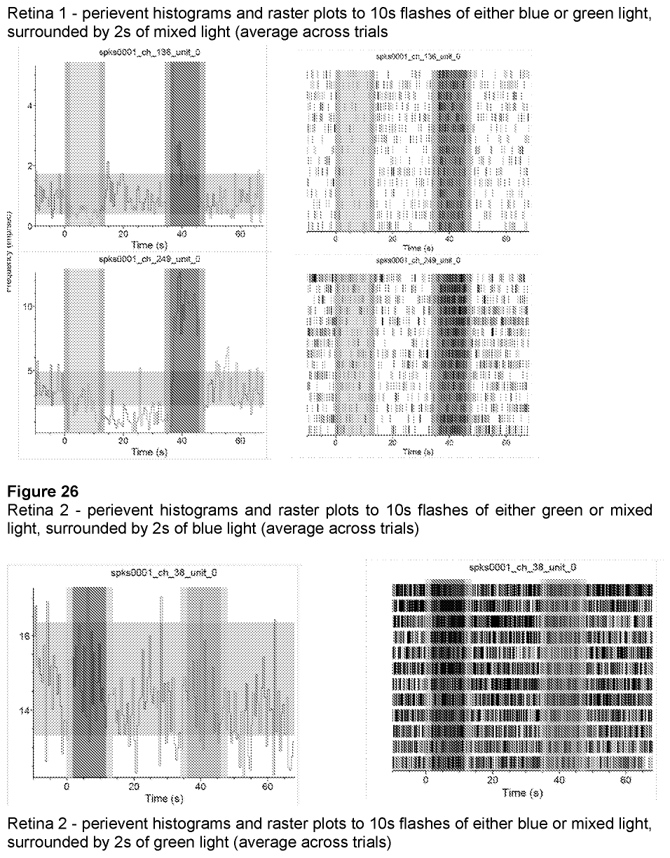

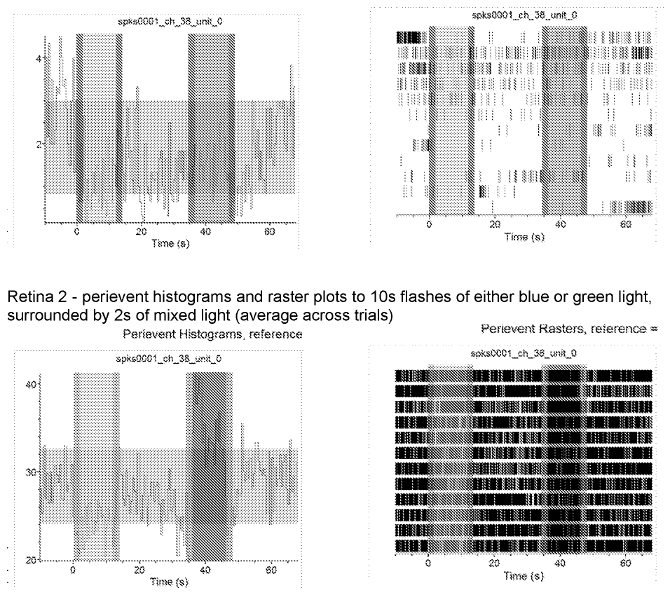

It was surprising to find that the sequential light stimuli enabled precise control of timing of onset and offset of excitation or inhibition. For example, exposing a Green ON unit to 10 s of green light resulted to sustained excitation. However, this could be rapidly switched off when the green light was immediately followed by 2 s of blue or mixed light. Similarly, the sustained inhibition shown by Green ON units in response to blue light could be reversed back to baseline spike firing levels by changing from blue to green or mixed light. This is consistent with the ability of different wavelengths to convert lamprey parapinopsin from active to inactive state, and vice versa. We were able to detect these responses to sequential light stimuli from two different retinas, suggesting they are an intrinsic property of retinal cells transduced with lamprey parapinopsin virus (FIG. 26).

The longer duration of these sequential light stimuli meant they could potentially trigger light responses from melanopsin-expressing intrinsically photosensitive retinal ganglion cells in the AAV-treated retinas. However, the ipRGC light responses to these stimuli are easy to distinguish from those driven by lamprey parapinopsin (FIG. 19 which shows two units with typical melanopsin responses to 10 s of either blue or green light surrounded by 2 s of mixed light. Top panel shows raster plots, bottom panel shows perievent spike firing rate histograms averaged across 15 trials). First, ipRGCs responses will have similar responses to all sequential light stimuli as the green and blue lights used here have similar effective photons for melanopsin. Second, melanopsin-driven responses have slower onset and remain sustained after light stimulus is complete, unlike the lamprey parapinopsin-driven responses which are switched off by changes in wavelength. Third, melanopsin does not cause inhibition of spike firing. This slow sustained excitation to both blue and green light was the only type of light response found in untreated rd1 retinas.

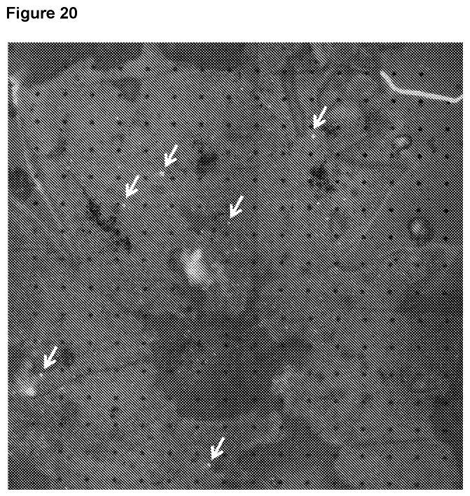

Fluorescence microscopy of retinal wholemounts on the multi-electrode array after recording demonstrated expression of mCherry reporter, confirming that viral transduction was successful and widely distributed across the retina (FIG. 20).

Methods

Animals

All experiments and care was conducted in accordance with the UK Animals (Scientific Procedures) Act (1986). Grm6^{+/Cre} (Morgan, C. W. et al, (2009). Proc Natl Acad Sci USA, 106 (45), 19174-8, doi: 10.1073/pnas.0908711106) rd1 mice on a mixed C3H×C57BI/6 background were used. These mice have Cre recombinase expressed under control of the Grm6 (mGlur6) promoter, resulting in restricted expression of Cre in rod bipolar cells. L7^{+/Cre} (Marino, S., et al. (2002). Development (Cambridge, England), 129(14), 3513-3522) rd1 transgenic mice on a mixed C3H×C57BI/6 background were also used. L7Cre mice have Cre cDNA inserted in exon 4 of the L7 gene, resulting in restricted expression of Cre recombinase in rod bipolar cells and small subset of ganglion cells in the retina (Ivanova et al., 2010). They also possess the Pde6b^{rd1} mutation Chang, et al (2002) Vision Research, 42(4), 517-525. https://doi.org/10.1016/S0042-6989(01)00146-8 and Pittler, S. J., & Baehr, W. (1991) Proceedings of the National Academy of Sciences of the United States of America, 88(19), 8322-8326. https://doi.org/10.1073/pnas.88.19.8322), which causes progressive retinal degeneration with vision loss complete once animals are over 80 days old. Mice were genotyped to confirm they do not possess the GRP179 point mutation (Peachey et al. (2012) American Journal of Human Genetics, 90(2), 331-339. https://doi.org/10.1016/j.ajhg.2011.12.006) that affects bipolar cell function. Mice were kept under a 12:12 light dark cycle with food and water provided ad libitum. Multi-electrode array recordings were conducted between 13 to 15 weeks after bilateral intraocular injection of adeno-associated virus at 9-10 weeks old.

Intravitreal Injections

Grm6^{Cre/+} rd1 or L7^{Cre/+} rd1 mice received bilateral intravitreal injections of lamprey parapinopsin virus (AAV2 4YF—ITR—DIO-CMV-LPPN-1D4-T2A-mCherry—WPRE- SV40 late polyA—ITR). The lamprey parapinopsin virus was packaged in an AAV2/2 capsid with four tyrosine to phenylalanine mutations (Petrs-Silva et al (2011) Molecular Therapy: The Journal of the American Society of Gene Therapy, 19(2), 293-301. https://doi.org/10.1038/mt.2010.234) to achieve efficient viral transduction of retinal cells, in particular bipolar cells. An 8 amino-acid C-1D4 tag (ETSQVAPA) was added to the C-terminus of the lamprey parapinopsin transgene (AB116380.1). The Lamprey parapinopsin-1D4 transgene (LPPN-1D4) was linked to a mCherry fluorescent reporter using a T2A sequence to ensure 1:1 co-expression of the two proteins. The inverted LPPN-1D4-T2A-mCherry open reading frame was flanked by two pairs of Lox sites (LoxP and Lox2272), so that in the presence of Cre recombinase, the transgene is inverted into the sense orientation and expression is driven by the constitutive CMV (cytomegalovirus) promotor. A woodchuck hepatitis virus post-transcriptional regulatory element (WPRE) and SV40 late polyA sequence were also included between ITRs to improve transgene expression. Virus was obtained from VectorBuilder.

Mice were anaesthetised by intraperitoneal injection ketamine (75 mg/kg body weight) and medetomidine (1 mg/kg body weight). Once anaesthetised, mice were positioned on a heat mat to prevent cooling. Pupils were dilated with 1% tropicamide eye drops (Bausch & Lomb) and a 13 mm coverslip was positioned on gel lubricant (Lubrithal) applied to the cornea. Between 2.2-2.5 ul of virus (1.12×10¹³ genomic counts per ml) was injected into the vitreous of each eye using a Nanofil 10 μl syringe (World Precision Instruments) using 35-gauge bevelled needle using a surgical microscope (M620 F20, Leica). All mice received bilateral injections. Anaesthesia was reversed by intraperitoneal injection of atipamezole (3 mg/kg body weight). During recovery, 0.5% bupivacaine hydrochloride and 0.5% chloramphenicol was applied topically to the injected eyes. Mice also received 0.25 ml of warm saline given by subcutaneous injection to aid recovery.

Multi-Electrode Array Recordings

Mice were dark adapted overnight. All following steps were performed under diffuse dim red light. Dark adapted mice were culled by cervical dislocation (approved Schedule 1 method). Enucleated eyes were placed in petri dish filled with carboxygenated (95% O₂/5% CO₂) Ames' media (supplemented with 1.9 g/L sodium bicarbonate, pH 7.4, Sigma Aldrich) and retinas dissected, with care taken to remove vitreous from inner retinal surface. Retinal wholemounts were then placed on glass coated metal harps (ALA Scientific Instruments), and positioned ganglion-cell side down on coated 256-channel multi-electrode arrays (MEA, Multi Channel Systems). Multi-electrode arrays were first incubated in fetal bovine serum overnight at 4° C., then coated with 0.1% polyethyleneimine (PEI) in borate buffer (pH8.4) for 1 hr at room temperature. PEI coating was then removed, and MEA washed 4-6 times with ddH₂O. PEI-coated MEAs were then air-dried and coated with 20 μg/ml laminin in fresh Ames' medium for 30-45 mins at room temperature (Egert, U., & Meyer, T. (2005), In S. Dhein, F. W. Mohr, & M. Delmar (Eds.), Practical Methods in Cardiovascular Research (pp. 432-453). https://doi.org/10.1007/3-540-26574-0_22, 2005; Lelong, et al (1992). Journal of Neuroscience Research, 32(4), 562-568. https://doi.org/10.1002/jnr.490320411). Laminin solution was removed before retina was positioned on the MEA. Once in place on the MEA, the retina was continuously perfused with carboxygenated Ames' media with 10 μM 9-cis retinal at 2-3 ml/min using a peristaltic pump (PPS2, Multi Channel Systems) and maintained at 34° C. using a water bath heater (36° C.), in-line perfusion heater (35° C.) and base plate heater (34° C.). Once positioned, retinas were perfused in dark for at least 45 mins before first light stimuli were applied. Data were sampled at 25 kHz using MC Rack software (Multi Channel Systems). A Butterworth 200 Hz high pass filter was applied to raw electrode data to remove low frequency noise. Amplitude threshold for spike detection was 4-4.5 standard deviations from baseline. Light stimuli were presented using a customised light engine (Thorlab LEDs). An Arduino Due microcontroller controlled by programmes written in LabVIEW (National Instruments) to control stimulus duration and intensity by altering LED output. Table 2 shows the intensity of light stimuli used for testing lamprey parapinopsin activity.

Immunohistochemistry and Fluorescence Microscopy.

Injected mice were culled by cervical dislocation (approved schedule 1 method). Eyes were removed and placed in 4% paraformaldehyde in phosphate buffered saline for 24 hrs at 4° C. Retinas were then dissected and permeabilised in 1% Triton-X in PBS for 3×10 mins at room temperature. Retinas were then blocked using 1% Triton-X in PBS with 10% normal donkey serum for 2-3 hours while shaking gently. Retinas were incubated in primary antibody (1:500 dilution of rabbit anti-mCherry antibody, Kerafast, catalogue no. EMU106, in 1% Triton-X in PBS with 2.5% donkey serum) overnight at room temperature. Retinas were washed in PBS with 0.2% Triton-X for 4×30 mins shaking gently. Retinas were incubated in secondary antibody (Goat anti-rabbit Alexa fluorophore 555, Abcam, catalogue no. ab150078, in 1% Triton-X in PBS with 2.5% donkey serum) for 3-4 hours at room temperature in the dark. Retinas were washed a further 4 times in PBS with 0.2% Triton-X for 30 mins each, then washed in ddH₂O for 10 mins. Retina was then mounted on a microscope slide using Prolong Gold anti-fade mountant and allowed to dry overnight at room temperature.

For images of retinal flatmounts on multi-electrode array, once recording was complete, media was drained from MEA chamber and metal harp removed. The entire MEA chamber was then placed on microscope stage and images of mCherry fluorescence from transduced cells in retinal wholemount were acquired.

Images of mCherry immunostaining and fluorescence were acquired using a Leica DM2500 microscope with DFC365 FX camera (Leica) and a CoolLED-pE300-white light source. Imaging software was Leica Application Suite Advanced Fluorescence6000. Images were acquired using Chroma ET Y3 filter set (excitation=545 nm, emission=610 nm). Global enhancements to image brightness and contrast were made using ImageJ software.

TABLE 2
Stimuli created by mixing different amount of monochromatic blue (405 nm) and
green (525 nm) light. Changing the ratio of these two wavelengths adjusts the proportion of
lamprey parapinopsin driven to inactive or active state.

| Relative % | Relative % | | | | | |
|---|---|---|---|---|---|---|
| of 405 nm | of 525 nm | Effective Log Photons/cm²/s | | | Relative Excitation | |
| in stimulus | in stimulus | Inactive state | Active state | Total | Inactive state | Active state |
| 0 | 100 | 10.60 | 14.01 | 14.01 | 0.00 | 1 |
| 50 | 50 | 13.49 | 13.85 | 14.01 | 0.30 | 0.70 |
| 100 | 0 | 13.79 | 13.61 | 14.01 | 0.60 | 0.40 |

Two types of light stimulus protocols were used. The first was flashes from dark and consisted of 10-20 cycles of 2 s 100% 405 nm, 20 s dark, 2 s 100% 525 nm, 20 s dark, 2 s 50% 405 nm 50% 525 nm, 20 s dark. The second was consecutive flashes, which consisted of 10-15 cycles of 2 s wavelength A, 10 s wavelength B, 2 s wavelength A, 20 s dark, 2 s wavelength A, 10 s wavelength C, 2 s wavelength A, 20 s dark (where wavelength A, B and C are any combination of 100% 405 nm, 100% 525 nm and 50% 525 nm 50% 405 nm). Each light stimulus protocol was separated by at least 20 mins of dark adaptation. Where possible, responses from intrinsically photosensitive retinal ganglion cells were also recorded by measuring activity to 5-10 cycles of 20 s white light (14 log photons) followed by 2 min dark.

1. A method of regulating Go protein activation in a cell, wherein the method comprises i) transforming the cell with a vector encoding a bistable opsin, wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light comprising the first and/or second wavelength, wherein the ratio of the two wavelengths of light regulates a Go protein activation.

2. A method according to claim 1 wherein the regulation of the Go protein comprises quantitative regulation.

3. A method according to any one of claim 1 or 2 wherein the method comprises ON-OFF regulation of the Go protein, by exposing the cell to either light of a first wavelength or light of a second wavelength.

4. A method according to any one of claims 1 to 3 wherein the method relates to regulation of a Go protein in a retinal cell, further wherein the bistable opsin is lamprey parapinopsin.

5. A method according to any one of claims 1 to 4 wherein the cell is an inner retinal cell, for example a bipolar cell, for example an ON or OFF bipolar cell.

6. A method of quantitative regulation of G protein activation in a cell, wherein the method comprises i) transforming the cell with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light comprising the first and/or second wavelength, wherein the ratio of the two wavelengths of light regulates the amplitude of a G protein signalling cascade.

7. A method according to claim 6 wherein the size of the G protein activation response is regulated by the ratio of (light of a first wavelength): (light of a second wavelength) used to illuminate the cell.

8. A method of regulating G protein activation in a cell, wherein the method comprises i) transforming the cell with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength wherein the ratio of the two wavelengths of light regulates a G protein signalling cascade; and wherein the time for G protein response to illumination is between 0.05 and 15 seconds.

9. A method according to claim 8 wherein the regulation of the G protein comprises quantitative regulation, preferably of a Go protein in a retinal cell, preferably wherein the bistable opsin is lamprey parapinopsin.

10. A method for quantitative regulation of the quantity of bistable opsin in an inactive and active state in a cell, wherein transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; wherein the method comprises illuminating the cell expressing the bistable opsin with light of a first wavelength and/or light of a second wavelength, and wherein the ratio of the two wavelengths of light regulates the amount of bistable opsin in the active state.

11. A method according to claim 10 wherein the method comprises regulation of the bistable opsin, by exposing the cell to either light of a first wavelength or light of a second wavelength.

12. A method according to claim 10 or 11 wherein the light is a combination of light of a first wavelength and light of a second wavelength.

13. A method according to any one of claims 10 to 12 comprising quantitative regulation of the ON-OFF response of a bistable opsin in a retinal cell, preferably wherein the bistable opsin is lamprey parapinopsin.

14. A method according to claim 13 wherein the retinal cell is an inner retinal cell, preferably a bipolar cell, preferably an ON or OFF bipolar cell.

15. A method of restoring vision in a subject by restoring a visual signal transduction response in a retinal cell in a subject, the method comprising i) transforming a retinal cell in the subject with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell by regulating the amplitude of G protein activation.

16. A method of treatment of retinal degeneration by introducing a visual signal transduction response in a retinal cell in a subject, the method comprising i) transforming a retinal cell in the subject with a vector encoding a bistable opsin wherein the bistable opsin has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; ii) expressing the bistable opsin in the transformed cell; iii) illuminating the transformed cell expressing the bistable opsin with light of a first wavelength and/or light of a second wavelength, wherein the ratio of the two wavelengths of light regulates the amplitude of the visual signal transduction response in the cell by regulating the amplitude of G protein activation.

17. A method according to claim 15 or 16 wherein the bistable opsin is lamprey parapinopsin.

18. A method according to any one of claims 15 to 17 wherein the retinal cell is a retinal ganglion cell, or an inner retinal cell, more suitably a bipolar cell, such as an ON or OFF bipolar cell.

19. A method according to any one of claims 15 to 18 wherein the subject is suffering from, or predisposed to, a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability; or Parkinsons disease, Alzheimer's disease, schizophrenia or heart disease.

20. A method according for the treatment of Parkinsons disease, Alzheimer's disease, schizophrenia and heart disease, wherein the method is as defined in any one of claims 1 to 14.

21. A method according for the treatment of a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability, wherein the method is as defined in any one of claims 1 to 14.

22. A method according to any one of claims 15 to 21 wherein the regulation of the G protein comprises quantitative regulation.

23. A method according to any one of claims 6 to 22 wherein the G protein is a Go or Gi protein.

24. A method according to any one of claims 6 to 10, and 15 to 23 wherein the method comprises ON-OFF regulation of the G protein, by exposing the cell to either light of a first wavelength or light of a second wavelength.

25. A method according to any one of the preceding claims wherein the first and second wavelength differ by at least 10 nm; and wherein where the light of the first and/or second wavelength is not a single wavelength, it is the wavelengths having the maximum output which differ from each other by at least 10 nm.

26. A method according to any one of the preceding claims wherein the light of a first wavelength is from 380-450 nm for transition from the inactive state to active state; and/or the light of the second wavelength is from 450-650 nm for transition from an active to inactive state.

27. A method according to any one of the preceding claims wherein the method comprises illuminating the cell with light of a first wavelength or light of a second wavelength for a time period from one or more milli-seconds, up to one or more hours, or during waking hours; preferably wherein the ratio of light of the first wavelength to light of the second wavelength may change one or more times during the time period.

28. A method according to claim 27 wherein the ratio changes every millisecond.

29. A nucleic acid vector comprising a nucleic acid encoding i) a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different; and ii) a promoter which is specific for expression in an inner retinal cell.

30. A nucleic acid vector according to claim 29 wherein the promoter is specific for bipolar cells, specifically ON bipolar cells.

31. A nucleic acid vector according to claim 29 or 30 wherein the promoter is grm6, a fragment thereof or a derivative thereof, and preferably is a synthetic promoter derived from a naturally occurring grm6 promoter.

32. A nucleic acid vector according to any one of claims 29 to 31 wherein the vector is a viral vector, preferably AAV.

33. A kit comprising i) a nucleic acid vector comprising a nucleic acid encoding a bistable opsin which has an inactive and an active state, and transition in both directions between inactive and active states are driven by light absorption; and wherein the bistable opsin in the inactive state is sensitive to a first wavelength of light for transition to an active state, and the bistable opsin in the active state is sensitive to a second wavelength of light for transition to an inactive state, the first and second wavelengths being different, and ii) a light source which emits light in the first wavelength and/or light in the second wavelength; preferably wherein the vector is as defined in any one of claims 29 to 32.

34. A nucleic acid vector or a kit according to any one of claims 29 to 32 for use in the treatment of Parkinsons disease, Alzheimer's disease, schizophrenia and heart disease; or for use in the treatment of a disease selected from the group consisting of a retinal dystrophy including a rod dystrophy, a rod-cone dystrophy, a cone-rod dystrophy, a cone dystrophy and a macular dystrophy; another forms of retinal or macular degeneration, an ischaemic conditions, uveitis and any other disease resulting from loss of photoreceptor ability.

35. A method, vector or kit according to any one of the preceding claims wherein the bistable opsin is a parapinopsin, for example lamprey parapinopsin, preferably encoded by the sequence of Genbank AB116380.1 or a variant or derivative thereof.

36. A method, vector or kit according to any one of the preceding claims wherein the sequence encoding a bistable opsin is provided in a viral vector, preferably an AAV vector.
